(19) United States
(12) Patent Application Publication
(10) Pub. No.: US 2024/0277835 A1
(43) Pub. Date: Aug. 22, 2024

(54) VACCINE

(71) Applicant: ASTRAZENECA AB, Södertälje (SE)

(72) Inventors: Joseph Richard FRANCICA, Gaithersburg, MD (US); Wade Stanton BLAIR, Gaithersburg, MD (US); Jason Paul LALIBERTE, Gaithersburg, MD (US); Yueh-Ming LOO, Gaithersburg, MD (US)

(21) Appl. No.: 18/417,379

(22) Filed: Jan. 19, 2024

Related U.S. Application Data
(60) Provisional application No. 63/480,754, filed on Jan. 20, 2023.

(51) Int. Cl. A61K 39/215; A61K 39/00; A61P 31/14; C12N 7/00

(52) U.S. Cl. CPC A61K 39/215; A61P 31/14; C12N 7/00; A61K 2039/53; A61K 2039/55; A61K 2039/55555; A61K 2039/70; C12N 2770/20022; C12N 2770/20034

(57) ABSTRACT

The present disclosure relates to methods of inducing a pan-sarbecoronavirus variant immune response for the treatment and prevention of coronavirus infections.

### CROSS-REFERENCE TO RELATED APPLICATIONS

This application claims benefit under 35 U.S.C. § 119(e) of U.S. Provisional Application No. 63/480,754, filed Jan. 20, 2023, which is incorporated by reference herein.

### REFERENCE TO SEQUENCE LISTING

This application incorporates by reference a Sequence Listing submitted electronically with the application as an XML file entitled “COFER-102-SubSequenceListing.xml” created on Mar. 5, 2024 and having a size of 54,936 bytes.

### FIELD

The present disclosure relates to immunogenic compositions useful for generating a pan-sarbecoronavirus immune response as part of a prime-boost vaccination regimen.

### BACKGROUND

A number of vaccines are approved for the treatment of COVID-19, but data suggests that the efficacy of vaccination can vary between variants. Since the first reports of SARS-COV-2, there has been the evolution of multiple variants of concern. Therefore, vaccines which target multiple strains of SARS-COV-2 may be of great benefit for immunisation against COVID-19.

However, a phenomenon known as “antigenic sin” has been observed. “Antigenic sin” is where dominant epitopes from an initial prime vaccination are recalled in a boost vaccination, even where the boost vaccine is directed against a different variant. This poses a challenge to achieving a broad neutralising response against multiple variants.

Nanoparticles displaying antigens have been proposed as vaccines to elicit broad neutralising responses against highly mutational viruses, such as sarbecoronavirus or influenza.

The premise of the strategy is that the virus-like particle (VLP) can display multiple variants of virus-derived antigens, such as SARS-COV-2. Displaying multiple antigens can generate immune responses against conserved epitopes, thereby limiting the probability of immunological escape by mutation.

The approach has been successfully employed in a number of in vivo, pre-clinical vaccination studies. For example, Boyoglu-Barnum et al (Nature, 592; 623-628 (2021)) shows quadrivalent HA-VLPs induce broad protection against flu. Cohen et al (Science, 377, eabq0839 (2022)) shows that a nanoparticle displaying 8 different sarbecoronavirus spike receptor binding domains protect against challenge by diverse sarbecoronaviruses in animal models. Thus, the approach is promising.

However, these examples of broad neutralisation responses are achieved using protein-based nanoparticle vaccines, which require expensive and complex manufacturing processes. This is in contrast to mRNA vaccines, which are generally cheaper to manufacture and have much more robust manufacturing processes, which is very advantageous when changes to vaccines are needed in response to immunological escape. The one draw-back associated with mRNAs is a general size limitation of the vaccine that can be administered. For example, it can be challenging to develop mRNA vaccines encoding up to eight different antigenic sequences, each of which are fused to multimerization elements that enable VLPs to form.

Thus, it is desirable to develop a vaccine that combines the advantages of both mRNA technology and broad neutralisation achieved with multivalent antigen nanoparticles.

### SUMMARY

The present disclosure relates to an immunogenic composition that is useful for generating a pan-sarbecoronavirus immune response as part of a prime-boost vaccination regimen. The composition comprises mRNA encoding a single spike protein from a sarbecoronavirus variant, together with the coding sequence of a multimerization unit, such that upon assembly in vivo a multimeric complex is formed.

Certain instances of the present disclosure are summarized below. This list is only exemplary and not exhaustive of all of the instances provided by this disclosure.
1. A method of inducing a pan-sarbecoronavirus variant immune response in an individual who has previously received one or more doses of a first SARS-COV-2 vaccine directed against a first sarbecoronavirus variant or variants (Variant 1), said method comprising administering to said individual one or more doses of a second SARS-COV-2 vaccine, wherein the second SARS-COV-2 vaccine comprises a mRNA encoding a single Spike (S) protein, or an immunogenic fragment or immunogenic variant thereof, derived from a sarbecoronavirus variant (Variant 2) that is different to Variant 1, wherein the S protein is encoded as a S protein-multimerization subunit fusion, and wherein the method induces a pan-variant immune response in the individual against sarbecoronavirus Variant 1 and Variant 2 and induces an immune response against one or more additional sarbecoronavirus variants that are different from Variant 1 and Variant 2.
2. A method according to clause 1, wherein the S protein-multimerization subunit fusion is a protein-ferritin subunit fusion.
3. A method according to clause 1 or clause 2, wherein the mRNA of the second SARS-COV-2 vaccine is formulated in a lipid nanoparticle (LNP).
4. A method according to any of clauses 1 to 3, wherein Variant 2 is SARS-COV-2 Omicron BA.4/5.
5. A method according to clause 4, wherein the second SARS-COV-2 vaccine comprises the mRNA sequence of SEQ ID NO. 11.
6. A method according to any of clauses 1 to 3, wherein Variant 2 is SARS-COV-2 Omicron XBB.1.5.
7. A method according to clause 6, wherein the second SARS-COV-2 vaccine comprises the mRNA sequence of SEQ ID NO. 17.
8. A method according to any of clauses 1 to 7, wherein the first SARS-COV-2 vaccine does not comprise a nucleic acid encoding a sarbecoronavirus S protein in the form of a fusion protein that is capable of assembling to form a nanoparticle in vivo.
9. A method according to any of clauses 1 to 8, wherein the method induces an immune response against one or more additional sarbecoronavirus variants that are mutationally diverse from Variant 1 and/or Variant 2.
10. A method according to any of clauses 1 to 9, wherein the second SARS-COV-2 vaccine is a monovalent vaccine.
11. A method according to any of clauses 1 to 9, wherein the second SARS-COV-2 vaccine is a bivalent vaccine comprising a further mRNA encoding a single Spike (S) protein, or an immunogenic fragment or immunogenic variant thereof, wherein the S protein is encoded as a S protein-multimerization subunit fusion and is derived from a sarbecoronavirus variant that is different to Variant 2 and is the same as or different to Variant 1.
12. A method according to any of clauses 1 to 11, wherein the first dose of the second SARS-CoV-2 vaccine is administered to the individual at least 4 months after administration of the final dose of the first SARS-COV-2 vaccine.
13. A method according to clause 12, wherein the first dose of the second SARS-COV-2 vaccine is administered to the individual at least 6 months after administration of the final dose of the first SARS-COV-2 vaccine.
14. An immunogenic composition comprising mRNA encoding a single Spike (S) protein, or an immunogenic fragment or immunogenic variant thereof, derived from a first sarbecoronavirus variant, for use in inducing a pan-sarbecoronavirus variant immune response in an individual who has previously received one or more doses of a first SARS-CoV-2 vaccine comprising or encoding an immunogen from a second sarbecoronavirus variant,
   wherein the S protein is encoded as a S protein-multimerization subunit fusion, and
   wherein the immunogenic composition is used to induce an immune response against the first and second sarbecoronavirus variants and against at least a third sarbecoronavirus variant.
15. An immunogenic composition for use according to clause 14, wherein the S protein-multimerization subunit fusion is a protein-ferritin subunit fusion.
16. An immunogenic composition for use according to clauses 14 or 15, wherein the mRNA is formulated in a lipid nanoparticle (LNP).
17. An immunogenic composition for use according to any of clauses 14 to 16, wherein the first sarbecoronavirus variant is SARS-COV-2 Omicron BA.4/5.
18. An immunogenic composition for use according to clause 17, wherein the mRNA comprises or consists of the mRNA sequence of SEQ ID NO. 11.
19. An immunogenic composition for use according to any of clauses 14 to 16, wherein the first sarbecoronavirus variant is SARS-COV-2 Omicron XBB.1.5.
20. An immunogenic composition for use according to clause 19, wherein the mRNA comprises or consists of the mRNA sequence of SEQ ID NO. 17.
21. An immunogenic composition for use according to any of clauses 14 to 20, wherein the first SARS-COV-2 vaccine does not comprise a nucleic acid encoding a sarbecoronavirus S protein in the form of a fusion protein that is capable of assembling to form a nanoparticle in vivo.
22. An immunogenic composition for use according to any of clauses 14 to 21, wherein the third sarbecoronavirus variant is mutationally diverse from the first and/or second sarbecoronavirus variants.
23. An immunogenic composition for use according to any of clauses 14 to 22, wherein the immunogenic composition is a monovalent composition.
24. An immunogenic composition for use according to any of clauses 14 to 22, wherein the immunogenic composition is a bivalent composition further comprising mRNA encoding a single Spike (S) protein, or an immunogenic fragment or immunogenic variant thereof, wherein the S protein is encoded as a S protein-multimerization subunit fusion and is derived from a sarbecoronavirus variant that is different to said first sarbecoronavirus variant and is the same as or different to said second sarbecoronavirus variant.

### DESCRIPTION OF THE FIGURES

Those of skill in the art will understand that the drawings, described below, are for illustrative purposes only. The drawings are not intended to limit the scope of the present teachings in any way.

FIG. 1A-C shows improved pan-variant immunogenicity in vaccine-experienced mice of an mRNA molecule according to the disclosure, encoding either a Wuhan D614G antigen fused to ferritin via a linker (group 2), a BA.4/5 antigen fused to ferritin via a linker (group 3), or combinations of the two (Wuhan D614G and BA.4/5 antigens fused to ferritin via a linker; group 4) compared to encoding two native spike antigens (Wuhan D614G and BA.4/5; group 1). FIG. 1a shows neutralizing antibody titres against BA.4/5 pseudovirus at day 180. FIG. 1b shows neutralising antibody titres 14 days after third vaccination (administered on day 231 as shown in FIG. 2). The number under each data bar indicates the geometric mean titres. FIG. 1c shows neutralizing antibody titres measured 14 days after a third vaccination with an mRNA molecule encoding either the native XBB.1.5 spike protein or the XBB.1.5 spike protein as a VLP antigen as measured among vaccine-experienced mice (similar study design as that depicted in FIG. 2). The number within each data bar indicates the geometric mean titres.

FIG. 2 shows a schematic of the boost vaccination mouse study design.

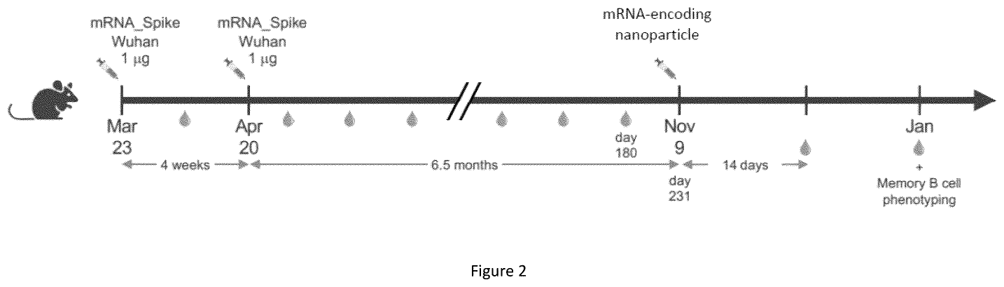

FIG. 3 shows neutralizing antibody titres against a panel of variant pseudoviruses as elicited by mRNA-native Delta or mRNA-VLP Delta vaccines in non-human primates 14 days after the second mRNA immunisation.

FIG. 4 shows the durability of neutralising antibody titres against the Delta variant pseudovirus as elicited by mRNA-native Delta or mRNA-VLP Delta vaccines in non-human primates out to 196 days after the first mRNA immunisation.

FIG. 5 shows the frequency of spike-specific memory B cells generated by mRNA-native Delta or mRNA-VLP Delta vaccines in non-human primates just prior to and 28 days after the second immunization.

FIG. 6 shows the enumeration of long-lived antibody secreting cells in the bone marrow of non-human primates 196 days after the first immunisation with mRNA-native Delta or mRNA-VLP Delta vaccine.

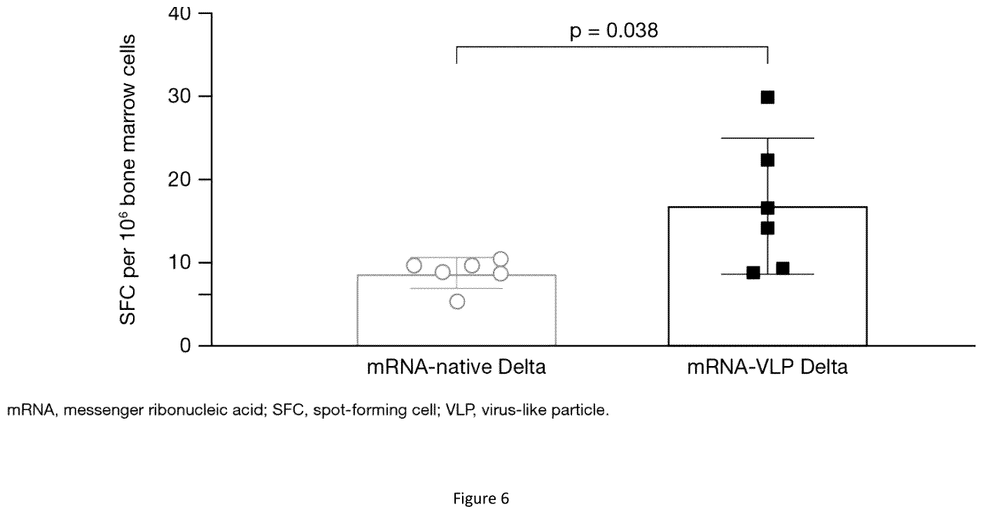

FIG. 7 shows the frequency spike-specific memory CD4+ T cells generated by mRNA-native Delta or mRNA-VLP Delta vaccines in non-human primates 28 days after the second immunization.

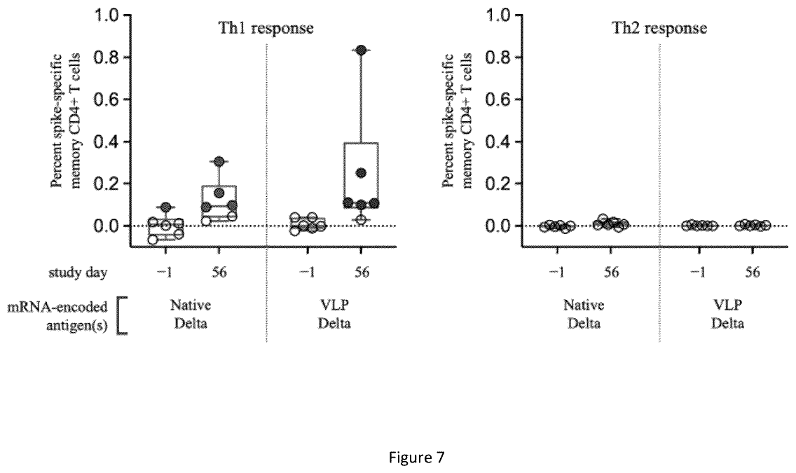

FIG. 8 shows a schematic of the boost vaccination non-human primate study design.

FIG. 9 shows neutralizing antibody titres against the ancestral Wuhan_D614G variant pseudovirus as elicited by mRNA-native XBB.1.5 or mRNA-VLP XBB.1.5 vaccines in vaccine-experienced non-human primates measured just prior to (day 246) and 14 days after the third immunization (day 260).

FIG. 10 shows neutralizing antibody titres against the Omicron BA.4/5 variant pseudovirus as elicited by mRNA-native XBB.1.5 or mRNA-VLP XBB.1.5 vaccines in vaccine-experienced non-human primates measured just prior to (day 246) and 14 days after the third immunization (day 260).

FIG. 11 shows neutralizing antibody titres against the Omicron XBB.1.5 variant pseudovirus as elicited by mRNA-native XBB.1.5 or mRNA-VLP XBB.1.5 vaccines in vaccine-experienced non-human primates measured just prior to (day 246) and 14 days after the third immunization (day 260).

FIG. 12 shows the fold rise in neutralizing antibody (nAb) titres 14 days after the third immunization against the different variant pseudoviruses tested amongst the vaccine-experienced non-human primates given either mRNA-native XBB.1.5 or mRNA-VLP XBB.1.5 vaccines.

### DETAILED DESCRIPTION

All references referred to are incorporated herein by reference in their entireties.

Many modifications and other instances of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific instances disclosed and that modifications and other instances are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

### Definitions

The term “nucleic acid sequence” is intended to encompass a polymer of DNA or RNA, i.e., a polynucleotide, which can be single-stranded or double-stranded and which can contain non-natural or altered nucleotides e.g. modified uridine. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides. Nucleic acids are typically linked via phosphate bonds to form nucleic acid sequences or polynucleotides, though many other linkages are known in the art (e.g. phosphorothioates, boranophosphates, and the like).

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulphide bond formation, glycosylation, lipidation, acetylation, phosphorylation, or any other manipulation or modification, such as conjugation with a labelling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in some aspects, the polypeptides can occur as single chains or associated chains.

“5′-untranslated region (5′-UTR)” has the usual meaning recognised by a skilled person. It is the region of a nucleic acid molecule located 5′ of a coding sequence and which is not translated into protein. A 5′-UTR usually starts with the transcriptional start site and end before the start codon of the coding sequence.

“3′-untranslated region (3′-UTR)” has the usual meaning recognised by a skilled person. It is the region of a nucleic acid molecule located 3′ of a coding sequence and which is not translated into protein. A 3′-UTR is usually 3′ of a coding sequence. If the molecule comprises a polyadenylation signal, the 3′-UTR is usually between the coding sequence and the polyadenylation signal.

“Coding sequence” is a continuous stretch of DNA or RNA beginning with a start codon (e.g., methionine (ATG or AUG)) and ending with a stop codon (e.g., TAA, TAG or TGA, or UAA, UAG or UGA). A coding sequence typically encodes a polypeptide. The coding sequences disclosed herein are operably linked to the 5′ and 3′ UTRs described herein.

“Messenger RNA (mRNA)” is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). Nucleic acids can comprise a region or regions of linked nucleosides. Such regions 5 may have variable backbone linkages. The linkages can be standard phosphodiester linkages, in which case the nucleic acids would comprise regions of nucleotides (a “nucleotide” refers to a nucleoside, including a phosphate group).

“Expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing) and (3) translation of an RNA into a polypeptide or protein.

The term “pharmaceutical composition” refers to a preparation which is in such form as to permit the biological activity of the active ingredient to be effective, and which contains no additional components which are unacceptably toxic to a subject to which the composition would be administered. The composition can be sterile.

As used herein, the terms “subject”, “individual” and “patient” are used interchangeably. The subject can be an animal. In some aspects, the subject is a mammal such as a non-human animal (e.g. cow, pig, horse, cat, dog, rat, mouse, monkey or other primate, etc.). In some aspects, the subject is a human.

As used in the present disclosure and claims, the singular forms “a”, “an”, and “the” include plural forms unless the context clearly dictates otherwise.

It is understood that wherever aspects are described herein with the language “comprising”, otherwise analogous aspects described in terms of “consisting of” and/or “consisting essentially of” are also provided. In this disclosure, “comprises”, “comprising”, “containing” and “having” and the like can mean “includes”, “including”, and the like; “consisting essentially of” or “consists essentially” are open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. The term “and/or” as used in a phrase such as “A and/or B” herein is intended to include both “A and B,” “A or B”, “A”, and “B”. Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

### Coronavirus Vaccines

The recent Covid-19 pandemic has led to an urgent need for improved vaccines that target coronaviruses of concern. To date several variants of SARS-COV-2 have been identified with some of the most infectious among these being the Delta and Omicron variants. Vaccines currently approved for the treatment of SARS-COV-2 all encompass stimulating immunity against the SARS-COV-2 spike protein. Although, a number of mutations in the receptor binding domain of the spike protein have been identified in new variants of SARS-COV-2, which is thought to have led to increased vaccine resistance in newly emerging variants (Zhao, J. et al., Environmental research, (2022), 206(112240)). Research estimates that current vaccines are around three to five-fold less potent against the Delta variant than the Alpha variant of SARS-COV-2 (Planas, D. et al., Nature, (2021), 596, 276-280).

Accordingly, there is an ongoing need for improved vaccines in general, including those useful in the prevention and treatment of coronaviruses.

The coronavirus virion comprises a large number of glycosylated spike (S) proteins projecting from the surface of the virion. These S proteins form trimer structures, and mediate virus entry into host cells, making it a primary target for vaccine design.

The coronavirus spike protein is 1273 amino acids in length and comprises a signal peptide, and S1 and S2 subunits. The S1 subunit contains a receptor-binding domain (RBD) that recognizes and binds to a specific host cell receptor, angiotensin-converting enzyme 2 (ACE2). The S2 subunit mediates viral cell membrane fusion.

Thus, in one instance, the nucleic acid molecule comprises a sequence encoding an S protein or antigenic fragment thereof. Upon delivery into a host cell, the S protein is translated and processed in the host cell, resulting in the presentation of a trimerized S protein on a host cell surface.

In some instances, the nucleic acid molecule comprises a sequence that encodes a CoV S protein and a ferritin protein, wherein the CoV S protein and ferritin assemble to form a nano antigen particle.

The S protein can be S in a pre-fusion conformation. Further, the S protein can comprise K986P and/or V987P mutations. These mutations stabilise the S protein in a pre-fusion conformation (Wrapp D et al. Science (2020), 367:1260-1263).

In one instance, the nucleic acid molecule of the present disclosure encodes an antigenic fragment thereof that is a receptor binding domain (RBD).

A vaccine according to the present disclosure comprises a nucleic acid molecule encoding a sarbecoronavirus (SARS-COV) antigen. The antigen may be an antigen as described anywhere herein. In one instance, the sarbecoronavirus antigen can be selected from any SARS-COV-2 antigen, or an immunogenic fragment thereof. In one instance, the nucleic acid molecule is an mRNA sequence encoding a SARS-COV-2 antigen selected from one or more of the following variants: Wuhan, Alpha, Beta, Delta and Omicron, optionally selected from subvariants BA.1, BA.2, BA.2.86, BA.3, BA.4/5, BQ.1, BQ.1.1, JN.1, XBB.1 and XBB.1.5. In one instance, the nucleic acid molecule is an mRNA sequence encoding a SARS-COV-2 Spike (S) protein, or immunogenic fragment thereof, selected from one or more of the following variants: Wuhan, Alpha, Beta, Delta and Omicron, optionally selected from subvariants BA.1, BA.2, BA.2.86, BA.3, BA.4/5, BQ.1, BQ.1.1, JN.1, XBB.1 and XBB.1.5.

The vaccine further comprises a nucleic acid sequence that encodes a multimerization unit. In one instance, the multimerization unit is a ferritin protein. The multimerization unit may be the scaffold for the antigen particle. In some instances, the ferritin is a Helicobacter pylori ferritin. In some instances, the nucleic acid sequence encoding the ferritin may be modified to remove native glycosylation sites. In some instances, the nucleic acid molecule comprises a sequence that encodes an antigenic protein and a ferritin protein, wherein the antigenic protein and ferritin assemble to form a nano antigen particle.

The vaccine according to the present disclosure further comprises a sequence encoding a linker. The linker can be encoded between the antigen and the multimerization unit, optionally a ferritin protein, such that the antigen is fused to the multimerization unit in the encoded molecule.

In one instance, the vaccine comprises RNA 5′- and 3′-UTR sequences and mRNA coding sequence.

### Multimerization Units

The vaccines described herein comprise nucleic acid molecules encoding fusion proteins that comprise vaccine antigens linked to multimerization units. In some instances, such multimerization units impart desired properties to an antigen encoded by the nucleic acid molecule. For example, the examples show that multimerization units improve the immunogenicity of an antigen (e.g., the COVID spike protein), as compared to the immunogenicity of the same antigen expressed without the multimerization units. Furthermore, the multimerization units provided herein improve the pan-variant response against an antigen. For example, a nucleic acid molecule provided herein comprising a coding sequence encoding a COVID spike protein-multimerization unit fusion protein, when administered as a boost vaccine, elicits a broader immune response against SARs-COV-2 variants compared to spike proteins alone.

In some instances, the multimerization unit is a protein that can self-assemble into protein nanoparticles that are highly symmetric, stable, and structurally organized, with diameters of 10-150 nm, a highly suitable size range for optimal interactions with various cells of the immune system. In some instances, viral proteins or virus-like particles (VLPs) can be used to form stable nanoparticle structures. Examples of such viral proteins are known in the art. For example, in some instances, the multimerization unit is a hepatitis B surface antigen (HBsAg). HBsAg forms spherical particles with an average diameter of ˜22 nm and which lacked nucleic acid and hence are non-infectious (Lopez-Sagaseta, J. et al. Computational and Structural Biotechnology Journal 14 (2016) 58-68). In some instances, the multimerization unit is a hepatitis B core antigen (HBcAg) self-assembles into particles of 24-31 nm diameter, which resembled the viral cores obtained from HEY-infected human liver. HBcAg produced in self-assembles into two classes of differently sized nanoparticles of 300 A and 360 A diameter, corresponding to 180 or 240 protomers. In some instances, the antigen is fused to HBsAG or HBcAG to facilitate self-assembly of nanoparticles displaying the antigen.

In some instances, the multimerization unit is selected from the following self-assembling proteins: ferritin, lumazine synthase and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The LS monomer is 150 amino acids long, and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 A diameter. Even LS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEES J. 2013, 280: 2097-2104).

### Linkers

Vaccines disclosed herein may comprise nucleic acids encoding fusion proteins. In such instances, each of the domains of the fusion protein (e.g., the antigen and the multimerization unit), may be separated by a coding sequence encoding a linker sequence. In some instances, the linker may be a glycine-serine linker.

In some instances, the glycine-serine linker has the following amino acid sequence: GSGGSG (SEQ ID NO: 4). In some instances, the glycine-serine linker is encoded by SEQ ID NO: 5.

The skilled person will appreciate that other art-recognized linkers may be suitable for use in the constructs of the disclosure (e.g., encoded by the nucleic acid molecules provided herein). The skilled person will likewise appreciate that other polycistronic constructs (nucleic acid molecules encoding more than one antigen/polypeptide separately within the same molecule) may be suitable for use as provided herein.

In various instances, a vaccine according to the disclosure comprises, in a 5′ to 3′ direction of transcription, a promoter, a 5′-UTR and a 3′-UTR flanking an antigen coding sequence and a polyadenylation signal.

In one instance, a vaccine described herein further comprises a 5′-cap structure, optionally a cap1 structure. Further suitable cap structures and approaches for generating suitable cap structures are disclosed in WO2017/053297 and Tusup et al., Design of in vitro Transcribed mRNA Vectors for Research and Therapy, Chim Int J Chem. 2019:73(5):391-394, both of which are hereby incorporated by reference. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; 35 G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-0 methyl-transferase to generate: m7G(5′)ppp(5′)G-2′-O-methyl. Cap 2 structure may be generated from the Cap 1 structure followed by the 2′-O-methylation of the 5′antepenultimate nucleotide using a 2′-0 methyl-transferase. Cap 3 structure may be generated from the Cap 2 structure followed by the 2′-O-methylation of the 5′-preantepenultimate nucleotide using a 2′-0 methyl-transferase. Enzymes may be derived from a recombinant source. Further suitable means for generating suitable cap structures are disclosed in WO2016/193226, which is hereby incorporated by reference.

In one instance, a vaccine of the disclosure comprises a promoter that is any promoter for a DNA-dependent RNA polymerase. For example, T7 (optionally comprising or consisting of the sequence TAATACGACTCACTATAAGG (SEQ ID NO: 15), T3, SP6 or Syn5 RNA polymerases.

In some instances, the vaccine disclosed herein comprises a polyadenylation signal (Poly A tail). The Poly A tail is a long sequence of adenine residues which lies at the 3′ end of the molecule. The role of the Poly A tail is two-fold. The Poly A tail is essential for translation, with Poly(A) binding proteins (PABP) recruiting translation factors to enhance translation levels. Furthermore, the Poly A tail increases the stability of a nucleic acid molecule by PABP binding poly(A) in mRNA and protecting it against exonuclease digestion. In mRNA, the poly A tail is also known to play a key role in the transport of mRNA from the nucleus to the ribosomes (Shlake, T., et al., RNA Biol., (2012), 9(11), 1319-1330). In one instance, the nucleic acid molecule of the disclosure comprises a Poly A tail of about 50 to about 500 adenosine nucleotides. For example, the poly A tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosines. In some instances, the poly A tail contains 50 to 250 adenosines. In some instances, the poly A tail contains 60 to 100 adenosines. In some instances, the poly A tail contains 70, 71, 72, 73, 74, 75, 76, 77, 78, 79 or 80 adenosines. In some instances, the poly A tail contains 77 adenosines. In some instances, the poly A tail contains 74 adenosines.

In one instance of the disclosure, the vaccine comprises a split Poly(A) tail. A split Poly(A) tail can comprise at least two adenosine containing elements, optionally of between 30 and 60 adenosines each, separated by a spacer optionally of between 1 and 25 nucleotides.

### Leader Sequences

In some instances, the antigen coding sequence disclosed herein comprises a leader sequence. A leader sequence may encode a signal peptide. In some instances, the signal peptide is fused to the expressed therapeutic protein. In such instances, the leader sequence and the gene of interest are within the same open reading frame (ORF).

Signal peptides, comprising the N-terminal 15-60 amino acids of proteins, are typically needed for the translocation across the membrane on the secretory pathway and control entry of most proteins to the secretory pathway. In eukaryotes, the signal peptide of a nascent precursor protein (pre-protein) directs the ribosome to the rough endoplasmic reticulum (ER) and initiates the transport of the growing peptide chain across it for processing. ER processing produces mature proteins, in which the signal peptide is typically cleaved by resident signal peptidases, at least for secreted proteins.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some instance, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids. In some instances, the signal peptide has the following sequence:

(SEQ ID NO: 8)
MPLLLLLPLLWAGALA.

### Nucleosides and Nucleotides

In some instances, the vaccine provided herein comprises a nucleic acid (preferably mRNA) that is not chemically modified and comprises the standard RNA nucleotides adenine (A), uracil (U), guanine (G) or cytosine (C).

In some instances, the vaccine comprises a nucleic acid comprising modified nucleotides. Many modified nucleotides are known in the art, such as disclosed in WO2007/024708, which is hereby incorporated by reference. Modifications can include either naturally occurring modifications or non-naturally occurring modifications. Modifications can include those at the sugar, backbone or nucleobase protein of the nucleotide and/or nucleoside as well known in the art.

In some instances, the nucleic acid molecules herein may include natural (i.e., standard) nucleotide or nucleoside, non-naturally or naturally occurring modified nucleotides or nucleosides, or any combination thereof.

In one instance, the RNA may comprise standard A, G and C nucleotides and modified U nucleotides.

In some instances, the nucleic acid molecule comprising the modified nucleoside or nucleotide (e.g., a “modified RNA nucleic acid molecule”) exhibits reduced immunogenicity in a cell or organism relative to an unmodified RNA nucleic molecule comprising the same sequence.

In some instances, modified nucleosides provided herein (e.g., RNA nucleic acid molecules such as mRNA) comprise N1-methyl-pseudouridine (m1ψ′), 1-ethyl-pseudouridine (e1ψ′), 5-methoxy-uridine (mo5U), 5-methyl-cytidine (m5C), and/or pseudouridine (ψ). In some instances, modified nucleotides in nucleic acid molecules (e.g., RNA nucleic acid molecules, such as mRNA) comprise 5-methoxymethyl uridine, 5-methylthio uridine, 1-methoxymethyl pseudouridine, 5-methyl cytidine, and/or 5-methoxy cytidine. In some instances, the RNA nucleic acid molecule includes a combination of at least two (e.g., 2, 3, 4 or more) of any of the aforementioned modified nucleobases.

In some instances, the nucleic acid molecule provided herein comprises N1-methyl-pseudouridine (m1ψ′) at one or more or all uridine positions of the nucleic acid molecule.

In some instances, the nucleic acid molecule comprises 5-methoxy-uridine (mo5U) at one or more or all uridine positions of the nucleic acid molecule.

In some instances, the nucleic acid molecule comprises from about 1% to about 100% modified nucleotides (either in relation to overall nucleotide content or in relation to one or more types of nucleotide (i.e., any one or more of A, G, U, T or C). In some instances, the nucleic acid molecule comprises any intervening percent of modified nucleotide content. For example, from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%. The remaining percentage is accounted for by unmodified A, G, U, T or C.

The nucleic acid molecules may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some instances, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some instances, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

In some instances, the nucleic acid molecules is an mRNA in which the uridine is replaced by a compound having a single unique structure. In some instances, the single unique structure is N1-methyl-pseudouridine. In some instances, the nucleic acid molecule comprises at least 5%, at least 10%, at least 25%, at least 50%, at least 80%, at least 90% or 100% N1-methyl-pseudouridine.

In one instance the mRNA comprises a modified nucleobase. In some instances, the modified nucleobase is modified adenine (A), cytosine (C), uracil (U) and guanine (G).

In one instance, the modified nucleobase is modified U. In some instances, the modified U is 1-methylpseudouridine (m1ψ′) and pseudouridine (ψ), such as is disclosed in WO2007/024708, which is hereby incorporated by reference.

In one instance, the nucleic acid molecules of the present disclosure comprise UTR sequences comprising 5-methoxy-uridine (mo5U) at one or more or all uridine positions of the nucleic acid molecule. The molecule can comprise at least 25% ratio of modified uridine to unmodified uridine, including 25% to 50%, or at least 50%.

The examples show that modifying uridine in a 5′-UTR sequence derived from the human CHIT1 5′-UTR and in a 3′-UTR sequence derived from the human citrate synthase (CS) 3′-UTR results in a particularly substantial increase in translation.

In one instance, the nucleic acid molecules of the present disclosure comprise sequences comprising N1-methyl-pseudouridine (m1ψ′) at one or more or all uridine positions of the nucleic acid molecule. The molecule can comprise at least 75% ratio of modified uridine to unmodified uridine, including 100%.

### Manufacturing

The vaccines of the present disclosure can be manufactured according to in vitro transcription. In vitro transcription of RNA is known in the art and is described in International Publication WO2014/152027, which is incorporated by reference herein in its entirety. In some instances, the RNA of the present disclosure is prepared in accordance with any one or more of the methods described in WO2018/053209 and WO2019/036682, each of which is incorporated by reference herein. In overview, a DNA template is generated, typically as a linearized plasmid, followed by in vitro transcription to synthesize the RNA, alongside or followed by capping.

The 5′ cap can be added by a multi-step enzymatic reaction or via co-transcription. In co-transcriptional capping, a cap analog, such as CleanCap® AG, is added directly to the in vitro transcription mixture. Alternatively, enzymatic capping using vaccinia virus capping enzyme is performed separately to the in vitro transcription.

Following purification, the mRNA product can be encapsulated in a lipid nanoparticle (LNP).

### Compositions

The present disclosure also provides a pharmaceutical composition comprising a nucleic acid molecule or LNP as defined anywhere herein and a pharmaceutical carrier.

The present disclosure further provides a composition comprising a first nucleic acid molecule according to the disclosure, wherein the disease-associated antigen is a Delta variant S protein. In one instance, the disease-associated antigen of a first nucleic acid molecule is a Wuhan variant S protein.

In one instance, the composition according to the disclosure can further include a second nucleic acid molecule encoding an Omicron variant S Protein, optionally variant BA.1, BA.2, BA.2.86, BA.3, BA.4/5, BQ.1, BQ.1.1, JN.1, XBB.1 or XBB.1.5.

In one instance, the second nucleic acid molecule encodes Omicron variant S Protein BA.4/5.

In one instance, the second nucleic acid molecule encodes Omicron variant S Protein XBB.1.5.

In all instances of the disclosure, the sequence encoding the antigen may be further optimized via mutation to increase protein stability (such as the structure of the CoV spike protein or the RBD), maximise protein translation and reduce unwanted side effects.

In some instances of the disclosure, the sequence encoding the antigen may be optimized via mutation to increase trimerized S protein stability and/or to ablate a furin cleavage site. In some instances of the disclosure, the disease-associated antigen is an antigenic fragment of the CoV S protein consisting of amino acid residues 1 to 1162 of the CoV S protein.

Said compositions may comprise an effective amount of the nucleic acid molecule as defined herein. An effective amount of the nucleic acid molecule to be employed therapeutically will depend, for example, upon the therapeutic objectives, the route of administration, and the condition of the patient. In one instance, the effective amount of the nucleic acid molecule as defined anywhere herein within the pharmaceutical composition is effective to treat or prevent a disease associated with coronavirus infection.

The composition is a pharmaceutically acceptable (e.g., physiologically acceptable) composition, which comprises a carrier, preferably a pharmaceutically acceptable (e.g., physiologically acceptable) carrier. The pharmaceutically acceptable carrier may include one or more excipients. Pharmaceutically acceptable excipients are known and include carriers, excipients, or stabilizers that are nontoxic to the cell or mammal being exposed thereto at the dosages and concentrations employed. Any suitable carrier can be used within the context of the disclosure, and such carriers are well known in the art. The choice of carrier will be determined, in part, by the particular site to which the composition may be administered and the particular method used to administer the composition. The physiologically acceptable excipient may be an aqueous pH buffered solution. Examples of physiologically acceptable excipients include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as Ethylenediaminetetraacetic acid (EDTA); sugar alcohols such as mannitol or sorbitol; salt-forming counterions such as sodium; and/or nonionic surfactants such as TWEEN™, polyethylene glycol (PEG), and PLURONICS™.

The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

The compositions can be administered intravenously. The composition can also be administered parenterally or subcutaneously.

Methods of administering a pharmaceutical composition as defined herein include, but are not limited to, parenteral administration (e.g., intradermal, intramuscular, intraperitoneal, intravenous and subcutaneous), epidural, and mucosal (e.g., intranasal and oral routes). In a specific example, a pharmaceutical composition is administered intranasally, intramuscularly, intravenously, or subcutaneously. The compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. Each dose may or may not be administered by an identical route of administration.

### Delivery System

Various delivery systems are known and can be used to administer a prophylactic or therapeutic agent (e.g., a nucleic acid molecule as disclosed herein), including, but not limited to, encapsulation in liposomes, microparticles, microcapsules, construction of a nucleic acid as part of a retroviral or other vector, etc. In addition, pulmonary administration can also be employed, e.g., by use of an inhaler or nebulizer, and formulation with an aerosolizing agent. The present disclosure relates to nucleic acid molecules that can be suitable for use as vaccine vectors.

Lipid nanoparticles (LNPs) may be used as a platform for vaccine vector delivery. LNPs may comprise ionizable cationic lipids, cholesterol, phospholipids as (such distearoylphosphatidylcholine), and polyethylene glycol (PEG)-lipid. Ionizable cationic lipids participate in nanoparticle packaging by interacting with negatively charged RNA molecules. Upon administration, LNPs are rapidly cleared from injected tissues, and are therefore less likely to induce inflammation and tissue damage.

Thus, in one instance of the present disclosure, the nucleic acid molecules as described anywhere herein are packaged into a delivery system. In one instance the delivery system is an LNP. Thus, the present disclosure also relates to LNPs comprising a nucleic acid molecule as described anywhere herein.

In one instance, the LNPs comprise nucleic acid molecules as described anywhere herein, wherein the nucleic acid molecule encodes an antigenic protein. In one instance, the LNPs comprise one or more nucleic acid molecules as described anywhere herein, wherein the molecules encode a CoV S protein.

### Uses of the Vaccine Composition

Following patient administration, the coding sequence will be transcribed and translated, in the case of a DNA sequence, and translated in the case of an RNA sequence, into the antigenic protein or fragment of an antigenic protein that it encodes for. The production of these antigenic proteins or antigenic protein fragments will stimulate an immune response, leading to the production of neutralising antibodies. Upon infection by a corresponding infectious agent, the presence of neutralising antibodies and memory B cells will increase the speed of the immune response, minimising the severity and length of symptom onset.

The vaccine vectors of the present disclosure may be used as a preventative therapy against a target antigen that causes disease. In one instance of the present disclosure, the vaccine vectors may be used in the prevention of CoV and, in particular, SARS-COV-2.

The vaccine vectors of the present disclosure may also be used as a treatment against a target antigen that has infected a subject. In one instance of the present disclosure, the vaccine vectors may be used in the treatment of CoV and, in particular, SARS-COV-2.

### Methods of Inducing a Pan-Sarbecoronavirus Variant Immune Response

According to a first aspect, the present disclosure provides a method of inducing a pan-sarbecoronavirus variant immune response in an individual who has previously received one or more doses of a first SARS-COV-2 vaccine directed against a first sarbecoronavirus variant or variants (Variant 1), said method comprising administering to said individual one or more doses of a second SARS-COV-2 vaccine, wherein the second SARS-COV-2 vaccine comprises a mRNA encoding a single Spike (S) protein, or an immunogenic fragment or immunogenic variant thereof, derived from a sarbecoronavirus variant (Variant 2) that is different to Variant 1, wherein the S protein is encoded as a S protein-multimerization subunit fusion, and wherein the method induces a pan-variant immune response in the individual against sarbecoronavirus Variant 1 and Variant 2 and induces an immune response against one or more additional sarbecoronavirus variants that are different from Variant 1 and Variant 2.

The phrase “pan-sarbecoronavirus immune response” refers to an immune response raised in response to challenge with multiple sarbecoronavirus variants. In a preferred instance the sarbecoronavirus variants are SARS-Cov-2 variants, including, but not limited to: Wuhan D614G, Alpha, Beta, Delta and Omicron, optionally subvariants BA.1, BA.2, BA.2.86, BA.3, BA.4/5, BQ.1, BQ.1.1, JN.1, XBB.1 and XBB.1.5.

In one instance Variant 2 is SARS-COV-2 Omicron BA.4/5. In one instance Variant 2 is SARS-CoV-2 Omicron XBB.1.5. In one instance Variant 1 is SARS-COV-2 Wuhan D614G. In one instance Variant 1 is SARS-COV-2 Delta.

In one instance the method induces an immune response against one or more additional sarbecoronavirus variants that are mutationally diverse from Variant 1 and/or Variant 2. As used herein, the term “mutationally diverse” refers to SARS-Cov2 variants that have separate and distinct mutational lineages. For example, mutationally diverse SARS-COV-2 variants may be designated according to the Pango lineage nomenclature. Pango lineages are designated to aid fine-scale tracking of SARS-COV-2. They represent clades within the phylogenetic tree defined by both at least one evolutionary event (nonsynonymous mutation, insertion/deletion or recombination event) and an event of epidemiological significance.

In one instance, the immune response is a neutralising response, determined by the presence of neutralising antibodies (NAbs). Methods for detecting the presence of NAbs in a sample obtained from an individual (including, for example, microneutralization assay, enzyme-linked immunosorbent assay (ELISA), and rapid lateral flow assay) will be apparent to the skilled person, and any suitable method may be used. Kuan-Ting Lui et al (Viruses (2022) 14(7) 1560) provides an overview of the International Standard for detecting SARS-COV-2 neutralizing antibodies, and the content of this publication is incorporated herein in its entirety.

The immune response is preferably a protective immune response, meaning that the immune response provides the individual with protection against infection or illness caused by SARS-CoV-2 variants. Protection may mean that the individual suffers no symptoms of infection, or that any symptoms of infection that are experienced following vaccination are less severe or shorter lasting than the would be experienced without vaccination.

Preferably the individual is a human, and may be an infant, child, adolescent or adult human.

The multimerization unit can be selected from the following self-assembling proteins: ferritin, lumazine synthase and encapsulin. In a preferred instance the protein-multimerization subunit fusion is a S protein-ferritin subunit fusion.

In one instance the second SARS-COV-2 vaccine comprises a further monovalent immunogenic composition comprising mRNA encoding a single Spike (S) protein, or an immunogenic fragment or immunogenic variant thereof, wherein the S protein is encoded as a S protein-multimerization subunit fusion and is derived from a sarbecoronavirus variant that is different to Variant 2 and is the same as or different to Variant 1.

In a one instance, the second SARS-COV-2 vaccine comprises mRNA formulated in a lipid nanoparticle (LNP).

In one instance, the second SARS-COV-2 vaccine comprises the mRNA sequence of SEQ ID No. 11.

In one instance, the second SARS-COV-2 vaccine comprises the mRNA sequence of SEQ ID No. 17.

In one instance, the first SARS-COV-2 vaccine comprises a protein antigen that is not provided in the form of a virus-like particle (VLP). In this instance, the first SARS-COV-2 vaccine does not comprise a nucleic acid encoding a sarbecoronavirus S protein in the form of a fusion protein that is capable of assembling to form a nanoparticle in vivo. As used herein “first” generally to a previous SARS-COV-2 vaccine that is compositionally distinct from the compositions disclosed herein. In other words, it describes a previous vaccine that encodes or delivers an S-protein antigen or immunogenic fragment or immunogenic variant thereof. Depending on the vaccination status of the subject, this could refer to the prime vaccine or a subsequent boost vaccine that is administered prior to the “second” vaccine disclosed herein.

In one instance the second SARS-COV-2 vaccine is a monovalent vaccine, meaning that the vaccine composition administered to the individual being vaccinated comprises mRNA encoding a single Spike (S) protein antigen, or an immunogenic fragment or immunogenic variant thereof.

In an alternative instance, the second SARS-COV-2 vaccine is a bivalent vaccine comprising a further mRNA encoding a single Spike (S) protein, or an immunogenic fragment or immunogenic variant thereof, wherein the S protein is encoded as a S protein-multimerization subunit fusion and is derived from a sarbecoronavirus variant that is different to Variant 2 and is the same as or different to Variant 1. In some instances, the further mRNA of the second SARS-COV2 vaccine comprises the mRNA sequence of SEQ ID NO: 9 or SEQ ID NO: 10.

One or more doses of each of the first and second SARS-COV-2 vaccines may be administered to the individual, as required as part of an approved prime-boost vaccination regimen. In one instance, the first dose of the second SARS-COV-2 vaccine is administered to the individual at least 4 months after administration of the final dose of the first SARS-COV-2 vaccine. In a further instance, the first dose of the second SARS-COV-2 vaccine is administered to the individual at least 6 months after administration of the final dose of the first SARS-COV-2 vaccine.

A second aspect of the disclosure provides an immunogenic composition comprising mRNA encoding a single Spike (S) protein, or an immunogenic fragment or immunogenic variant thereof, derived from a first sarbecoronavirus variant, for use in inducing a pan-sarbecoronavirus variant immune response in an individual who has previously received one or more doses of a first SARS-COV-2 vaccine comprising or encoding an immunogen from a second sarbecoronavirus variant, wherein the S protein is encoded as a S protein-multimerization subunit fusion, and wherein the immunogenic composition is used to induce an immune response against the first and second sarbecoronavirus variants and against at least a third sarbecoronavirus variant.

The phrase “pan-sarbecoronavirus immune response” refers to an immune response raised in response to challenge with multiple sarbecoronavirus variants. In one instance the sarbecoronavirus variants are SARS-Cov-2 variants, including, but not limited to: Wuhan D614G, Alpha, Beta, Delta and Omicron, optionally BA.1, BA.2, BA.2.86, BA.3, BA.4/5, BQ.1, BQ.1.1, JN.1, XBB.1 and XBB.1.5.

In one instance the first sarbecoronavirus variant is SARS-COV-2 Omicron BA.4/5. In one instance the first sarbecoronavirus variant is SARS-COV-2 Omicron XBB.1.5. In one instance the second variant is SARS-COV-2 Wuhan D614G. In one instance Variant 1 is SARS-COV-2 Delta.

Preferably the third sarbecoronavirus variant is mutationally diverse from the first and/or second sarbecoronavirus variants. As used herein, the term “mutationally diverse” refers to SARS-Cov2 variants that have separate and distinct mutational lineages. For example, mutationally diverse SARS-COV-2 variants may be designated according to the Pango lineage nomenclature. Pango lineages are designated to aid fine-scale tracking of SARS-COV-2. They represent clades within the phylogenetic tree defined by both at least one evolutionary event (nonsynonymous mutation, insertion/deletion or recombination event) and an event of epidemiological significance.

In one instance, the immune response is a neutralising response, determined by the presence of neutralising antibodies (NAbs). Methods for detecting the presence of NAbs in a sample obtained from an individual (including, for example, microneutralization assay, enzyme-linked immunosorbent assay (ELISA), and rapid lateral flow assay) will be apparent to the skilled person, and any suitable method may be used. Kuan-Ting Lui et al (Viruses (2022) 14(7) 1560) provides an overview of the International Standard for detecting SARS-COV-2 neutralizing antibodies, and the content of this publication is incorporated herein in its entirety. The immune response is preferably a protective immune response, meaning that the immune response provides the individual with protection against infection or illness caused by SARS-CoV-2 variants. Protection may mean that the individual suffers no symptoms of infection, or that any symptoms of infection that are experienced following vaccination are less severe or shorter lasting than the would be experienced without vaccination.

Preferably the individual is a human, and may be an infant, child, adolescent or adult human. In one instance, the S protein-multimerization subunit fusion is a protein-ferritin subunit fusion. In one instance the mRNA is formulated in a lipid nanoparticle (LNP).

In another instance, the mRNA comprises or consists of the mRNA sequence of SEQ ID NO. 11.

In another instance, the mRNA comprises or consists of the mRNA sequence of SEQ ID NO. 17.

In another instance, the first SARS-COV-2 vaccine comprises a protein antigen that is not provided in the form of a virus-like particle (VLP). In this instance, the first SARS-COV-2 vaccine does not comprise a nucleic acid encoding a sarbecoronavirus S protein in the form of a fusion protein that is capable of assembling to form a nanoparticle in vivo.

In one instance the immunogenic composition is a monovalent composition, meaning that it comprises mRNA encoding a single Spike (S) protein antigen, or an immunogenic fragment or immunogenic variant thereof.

In an alternative instance, the immunogenic composition is a bivalent composition comprising a further mRNA encoding a single Spike (S) protein, or an immunogenic fragment or immunogenic variant thereof, wherein the S protein is encoded as a S protein-multimerization subunit fusion and is derived from a sarbecoronavirus variant that is different to the first sarbecoronavirus variant and is the same as or different to said second sarbecoronavirus variant. In some instances, the further mRNA of the bivalent composition comprises the mRNA sequence of SEQ ID NO: 9 or SEQ ID NO: 10.

### EXAMPLES

The following examples further illustrate the disclosure but should not be construed as in any way limiting its scope.

### Example 1—Efficacy of mRNA Vaccine Vectors Comprising Antigen-Linker-Ferritin Sequences

The overall purpose of this study was to determine the immunogenicity of a candidate SARS-CoV-2 mRNA vaccine in mice. The mRNA vaccine encoded a stabilised Spike (S) protein-ferritin subunit fusion protein that, when expressed, assembles into a nanoparticle for high concentration antigen display.

### Methods

### Mouse Study 1

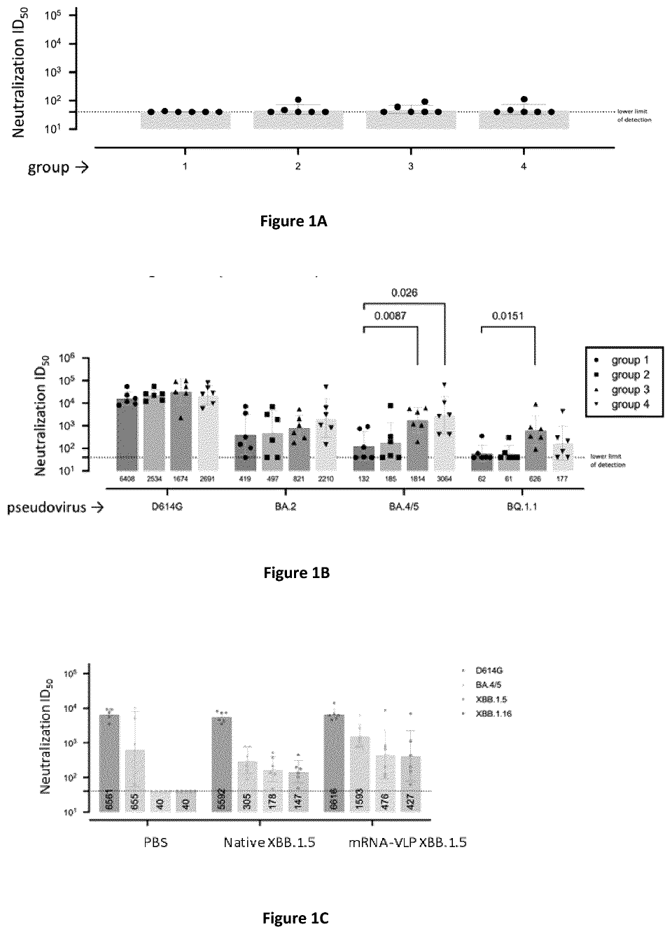

Groups of naïve BALB/c mice (n=6 per group) were administered LNP-formulated mRNA vaccines encoding the Wuhan D614G spike protein twice intramuscularly 28 days apart as 50 μl injections in the thigh muscle. After approximately 150 days, sera were collected and evaluated for neutralizing antibody levels against the BA.4/5 variant using a SARS-COV-2 pseudovirus-based neutralization assay (FIG. 1a).

After approximately 200 days, mice received a third immunization of LNP-formulated mRNA vaccines encoding the nanoparticles disclosed herein. 14 days later sera were collected and evaluated for neutralizing antibody levels against a panel of SARS-COV-2 variants employing a SARS-COV-2 pseudovirus-based neutralization assay (FIG. 1b).

At the day 200 point, mice were split into 4 groups and administered one or two mRNAs encoding antigens as specified in the following Table 1. Groups 1 and 4 were administered bivalent vaccines encoding both the Wuhan ancestral and BA. 4/5 Spike (S) proteins. Group 4 vaccines encoding the antigens as ferritin-linker fusion proteins (Virus-like particles, i.e., nanoparticles or “VLPs”). Alternatively, Groups 2 and 3 were administered vaccines encoding monovalent antigens (Group 2-Wuhan S protein, Group 3-BA. 4/5 S protein), both as VLPs.

TABLE 1

| | | |
|---|---|---|
| Group 1 | mRNA | Wuhan D614G FL Spike |
| | mRNA | BA. 4/5 FL Spike |
| Group 2 | mRNA | Wuhan D614G VLP |
| Group 3 | mRNA | BA. 4/5 VLP |
| Group 4 | mRNA | Wuhan D614G VLP |
| | mRNA | BA. 4/5 VLP |

### Mouse Study 2

Groups of naïve BALB/c mice (n=6 per group) were administered LNP-formulated mRNA vaccines encoding the Wuhan D614G spike protein twice intramuscularly 28 days apart as 50 μl injections in the thigh muscle.

After approximately 300 days, mice were divided into three groups. Group 1 was administered PBS, group 2 was administered a monovalent vaccine encoding the Omicron XBB.1.5 spike protein, and group 3 was administered a monovalent vaccine encoding the Omicron XBB.1.5 spike protein formatted as VLPs.

14 days later sera were collected and evaluated for neutralizing antibody levels against a panel of SARS-COV-2 variants employing a SARS-COV-2 pseudovirus-based neutralization assay (FIG. 1c).

### Results

The mouse study 1 modelling a boost vaccination campaign shows that VLP-based vaccines of groups 3 and 4 are more capable of overcoming antigenic sin experienced by the mice administered Group 1 vaccine, where the neutralising antibody response is dominated by ancestral variants (e.g., Wuhan “D614G”), used as inoculants in the original “prime” vaccination campaign. This is likely due to the mice originally being challenged with the Wuhan D614G S protein in the first vaccination campaign, and hence the memory recall of antibody pools neutralising this S protein dominates the Group 1 vaccine response.

Group 1 (and Group 2) mice show minimal neutralisation responses against BA. 4/5 (FIG. 1b), despite Group 1 animal being challenged with BA. 4/5 S protein encoding mRNA. By contrast, Groups 3- and 4-vaccinated mice show a 14 and 23-fold increase in mean neutralisation responses to BA 4/5 compared to Group 1 mice. Furthermore, Groups 3 and 4 mice also show low level neutralisation responses against the emerging variant of concern BQ.1.1, whereas no response is detected against this variant in Group 1 or 2 mice.

Similar results were obtained in mouse study 2, wherein group 3 mice show a 5.2-fold increase in mean neutralisation responses to BA.4/5, a 2.7-fold increase in mean neutralisation responses to XBB.1.5, and a 2.9-fold increase in mean neutralisation responses to XBB.1.16 compared to group 2 mice.

Overall, the data therefore suggests that broadly neutralising responses are obtainable from a single vaccine construct when mRNA is used to launch a VLP antigen. This may lead to better efficacy against multiple existing, and possibly future, variants of concern, without the requirement for boosters to be independently generated each time a novel dominant variant emerges.

### Example 2—Immunogenicity of mRNA Vaccine Vectors Comprising Antigen-Linker-Ferritin Sequences in Non-Human Primates

The overall purpose of this study was to determine the immunogenicity of the candidate SARS-CoV-2 mRNA vaccine in non-human primates (NHPs).

### Methods

SARS-COV-2 seronegative NHPs were immunised with two 10 μg doses (separated by 4 weeks) of either mRNA-native Delta vaccine or mRNA-VLP Delta vaccine. Neutralising antibody titres were measured two weeks after the second immunisation (Day 42) against a panel of SARS-COV-2 reporter viruses (ancestral D614G, Delta, BA.1, BA.2, or BA.4/5), and approximately 6.5 months after the first immunisation against the Delta reporter virus. The frequency of long-lived antibody secreting cells in bone marrow biopsies of vaccinated NHPs was measured using a B-cell ELISpot assay with recombinant Delta antigen.

### Results

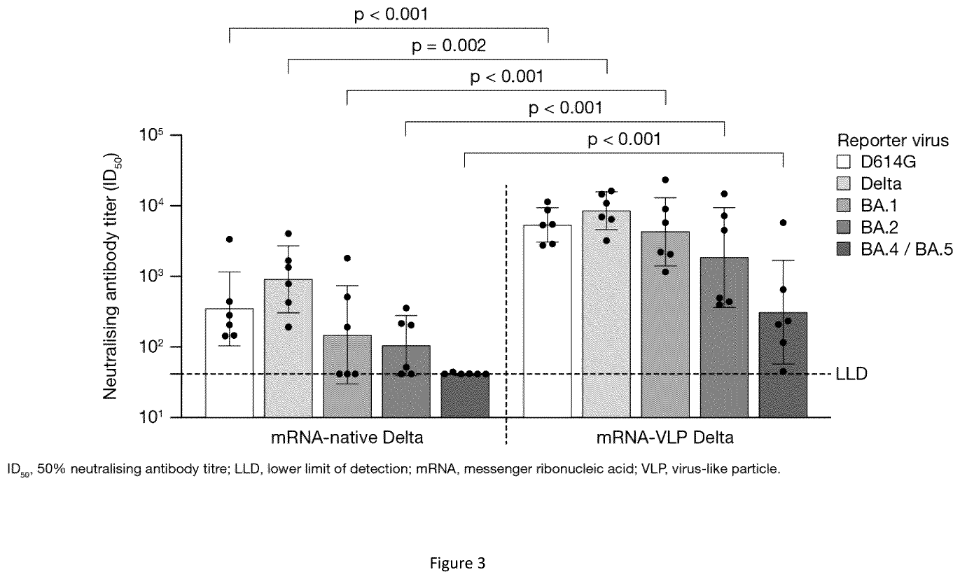

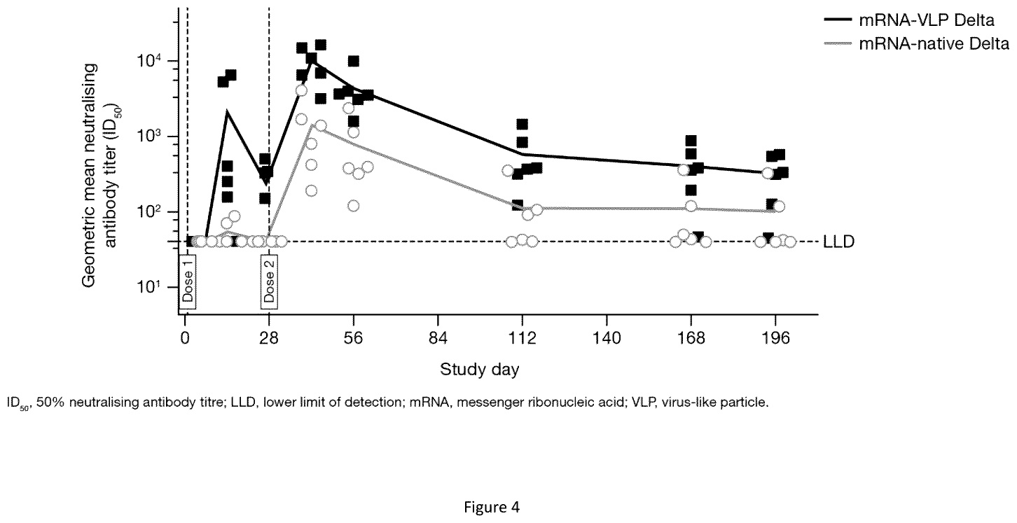

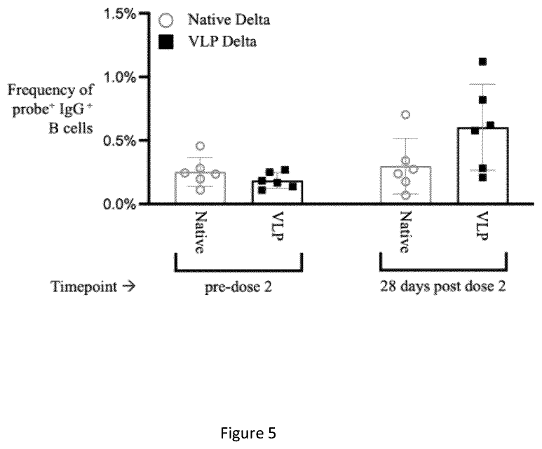

Two weeks after the second immunisation, the mRNA-VLP Delta vaccine elicited neutralising antibodies against D614G, Delta, BA.1, BA.2, or BA.4/5 reporter viruses with geometric mean titres (GMTs) 15-, 9-, 29-, 18-, and 7-fold higher, respectively, than those elicited by the mRNA-native Delta vaccine (all p≤0.002; FIG. 3). Moreover, 196 days after the first administration, neutralising antibody titres were 3.5-fold higher with mRNA-VLP Delta vs mRNA-native Delta (GMTs 236 and 68, respectively; FIG. 4). The mRNA-VLP Delta vaccine also induced a statistically significant 2-fold increase in long-lived antibody secreting cells vs the mRNA-native Delta vaccine (16.9 vs 8.8 per 106 bone marrow cells; FIG. 5).

Overall, the data confirms that broadly neutralising responses are obtainable from a single vaccine construct when mRNA is used to launch a VLP antigen, as observed in Example 1. The data also demonstrates that mRNA-VLP Delta vaccine elicits a more potent and broader neutralising antibody response vs the mRNA-native Delta vaccine in NHPs. Additionally, the mRNA-VLP Delta vaccine maintained higher neutralising antibody titres for ≥6 months and generated increased numbers of long-lived antibody secreting cells vs the mRNA-native Delta vaccine.

### Example 3—Boosting Capacity of mRNA Vaccine Vectors Comprising Antigen-Linker-Ferritin Sequences in Vaccine-Experienced Non-Human Primates

The overall purpose of this study was to determine the boosting capacity of the candidate SARS-COV-2 mRNA vaccine in vaccine-experienced non-human primates (NHPs).

### Methods

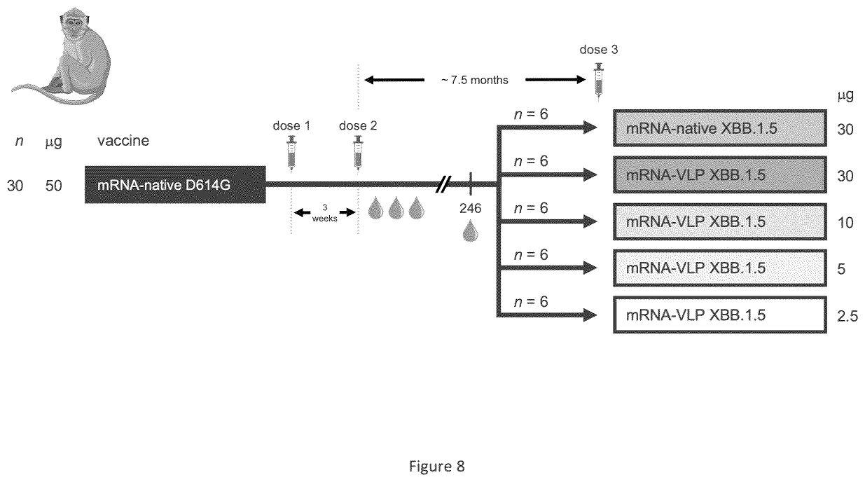

NHPs were first immunised with two doses (separated by 3 weeks) of mRNA-native ancestral Wuhan_D614G vaccine to establish baseline anti-spike immunity. Animals were then allowed to rest for approximately 7.5 months at which point they were administered a third, booster vaccination of either mRNA-native XBB.1.5 or mRNA-VLP XBB.1.5 (FIG. 8). Neutralising antibody titres were measured just prior to (day 246) and 14 days after (day 260) the third booster immunisation against a panel of SARS-COV-2 reporter viruses (ancestral Wuhan_D614G, Omicron BA.4/5, and Omicron XBB.1.5).

### Results

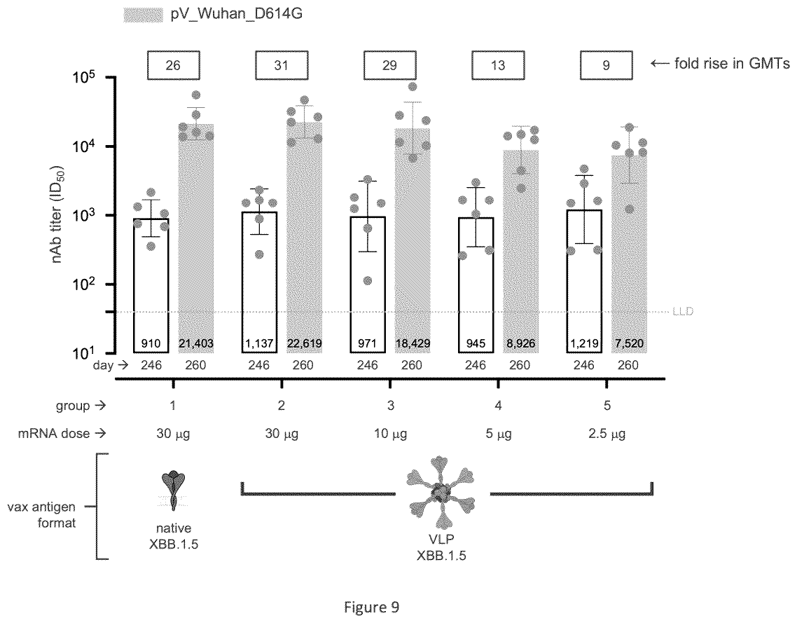

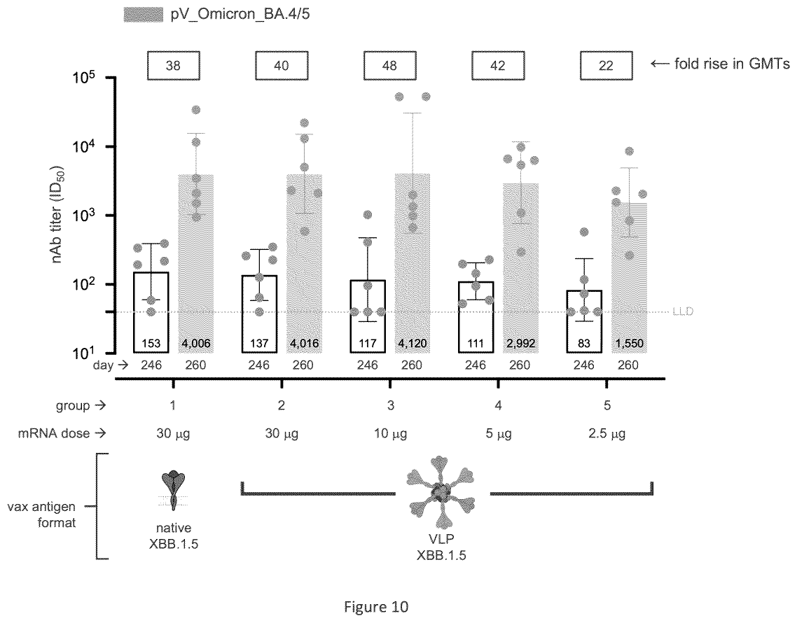

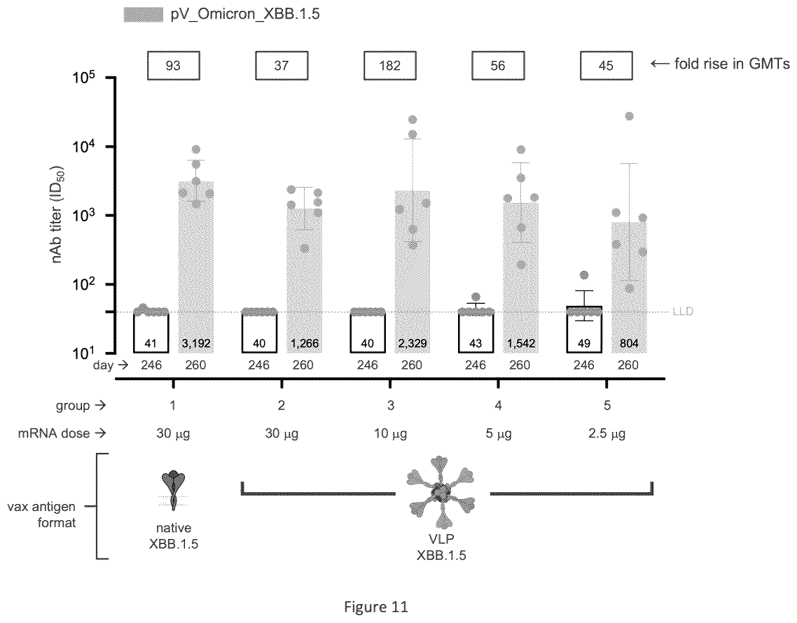

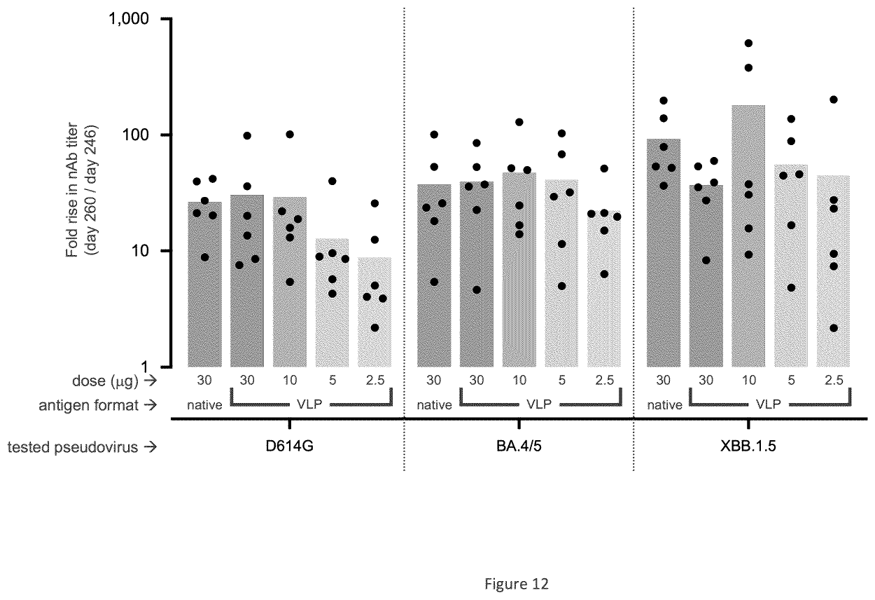

Two weeks after the third immunisation, the 30 μg dose of mRNA-VLP XBB.1.5 vaccine elicited neutralising antibodies against D614G, BA.4/5, and XBB.1.5 reporter viruses with geometric mean titres (GMTs) comparable to those titres elicited by the 30 μg dose of mRNA-native XBB.1.5 vaccine (FIGS. 9-12). Moreover, the animals immunized with either 10 or 5 μg of mRNA-VLP XBB.1.5 vaccine elicited neutralising antibody titres comparable to those induced by the 30 μg doses of mRNA-native XBB.1.5 or mRNA-VLP XBB.1.5 vaccines (FIGS. 9-12).

Overall, the data confirms that broadly neutralising responses are achieved by a single, low booster dose of mRNA-VLP vaccine when mRNA is used to launch a VLP antigen.

Sequences

SARS-CoV-2 Wuhan D614G Spike Protein-linker-ferritin RNA sequence (SEQ
ID NO: 1)
AUGCCCCUCCUUCUCCUUCUUCCCCUCCUUUGGGCUGGAGCGCUGGCCUCUCAGUG
UGUAAAUCUCACCACAAGAACCCAGCUGCCCCCUGCCUAUACCAAUUCCUUCACACG
GGGCGUGUACUAUCCCGACAAGGUGUUUAGAUCUAGCGUGCUGCACUCCACACAGG
AUCUGUUUCUGCCUUUCUUUUCUAACGUGACCUGGUUCCACGCCAUCCACGUGAGC
GGCACCAAUGGCACAAAGAGGUUCGACAAUCCAGUGCUGCCCUUUAACGAUGGCGU
GUACUUCGCCUCCACCGAGAAGUCUAACAUCAUCCGCGGCUGGAUCUUUGGCACCA
CACUGGACAGCAAGACACAGUCCCUGCUGAUCGUGAACAAUGCCACCAACGUGGUCA
UCAAGGUGUGCGAGUUCCAGUUUUGUAAUGAUCCAUUCCUGGGCGUGUACUAUCAC
AAGAACAAUAAGUCUUGGAUGGAGAGCGAGUUUCGGGUGUAUUCCUCUGCCAACAA
UUGCACAUUUGAGUACGUGUCCCAGCCCUUCCUGAUGGACCUGGAGGGCAAGCAGG
GCAAUUUCAAGAACCUGCGGGAGUUCGUGUUUAAGAAUAUCGAUGGCUACUUCAAGA
UCUACUCCAAGCACACCCCCAUCAACCUGGUGCGGGACCUGCCACAGGGCUUCUCU
GCCCUGGAGCCACUGGUGGAUCUGCCCAUCGGCAUCAACAUCACCCGGUUUCAGAC
ACUGCUGGCCCUGCACAGAAGCUACCUGACACCAGGCGACAGCUCCUCUGGAUGGA
CCGCAGGAGCAGCAGCCUACUAUGUGGGCUAUCUGCAGCCCCGGACCUUCCUGCUG
AAGUACAACGAGAAUGGCACCAUCACAGACGCCGUGGAUUGCGCCCUGGAUCCCCU
GUCUGAGACAAAGUGUACACUGAAGAGUUUUACCGUGGAGAAGGGCAUCUAUCAGA
CAAGCAAUUUCAGGGUGCAGCCUACCGAGUCCAUCGUGCGCUUUCCCAAUAUCACAA
ACCUGUGCCCUUUUGGCGAGGUGUUCAACGCCACCAGAUUCGCCAGCGUGUACGCC
UGGAAUAGGAAGCGCAUCUCCAACUGCGUGGCCGACUAUUCUGUGCUGUACAACAG
CGCCUCCUUCUCUACCUUUAAGUGCUAUGGCGUGAGCCCCACAAAGCUGAAUGACC
UGUGCUUUACCAACGUGUACGCCGAUUCCUUCGUGAUCAGGGGCGACGAGGUGCGC
CAGAUCGCACCAGGACAGACAGGCAAGAUCGCAGACUACAAUUAUAAGCUGCCUGAC
GAUUUCACCGGCUGCGUGAUCGCCUGGAACUCUAACAAUCUGGAUAGCAAAGUGGG
CGGCAACUACAAUUAUCUGUACCGGCUGUUUAGAAAGUCUAAUCUGAAGCCAUUCGA
GCGGGACAUCUCCACAGAGAUCUACCAGGCCGGCUCUACCCCCUGCAAUGGCGUGG
AGGGCUUUAACUGUUAUUUCCCUCUGCAGAGCUACGGCUUCCAGCCAACCAACGGC
GUGGGCUAUCAGCCCUACAGAGUGGUGGUGCUGUCUUUUGAGCUGCUGCACGCACC
UGCAACAGUGUGCGGACCAAAAAAGAGCACCAAUCUGGUGAAGAACAAGUGCGUGAA
CUUCAACUUCAACGGCCUGACCGGAACAGGCGUGCUGACCGAGUCCAACAAGAAGU
UCCUGCCUUUUCAGCAGUUCGGCAGGGACAUCGCAGAUACCACAGACGCCGUGCGC
GACCCUCAGACCCUGGAGAUCCUGGAUAUCACACCAUGCUCCUUCGGCGGCGUGUC
UGUGAUCACACCAGGCACCAAUACAAGCAACCAGGUGGCCGUGCUGUAUCAGGGCG
UGAAUUGUACCGAGGUGCCAGUGGCAAUCCACGCAGAUCAGCUGACCCCUACAUGG
CGGGUGUACUCUACCGGCAGCAACGUGUUCCAGACAAGAGCCGGAUGCCUGAUCGG
AGCAGAGCACGUGAACAAUAGCUAUGAGUGCGACAUCCCUAUCGGCGCCGGCAUCU
GUGCCUCCUACCAGACCCAGACAAACUCCCCAGGGUCUGCAUCAUCUGUGGCAAGC
CAGUCCAUCAUCGCCUAUACCAUGAGCCUGGGCGCCGAGAAUUCCGUGGCCUACUC
CAACAAUUCUAUCGCCAUCCCUACCAACUUCACAAUCUCCGUGACCACAGAGAUCCU
GCCAGUGAGCAUGACCAAGACAUCCGUGGACUGCACAAUGUAUAUCUGUGGCGAUU
CCACCGAGUGCUCUAACCUGCUGCUGCAGUACGGCAGCUUUUGUACCCAGCUGAAU
CGCGCCCUGACAGGAAUCGCAGUGGAGCAGGAUAAGAACACACAGGAGGUGUUCGC
CCAGGUGAAGCAGAUCUACAAGACCCCACCCAUCAAGGACUUUGGCGGCUUCAAUUU
UUCCCAGAUCCUGCCCGAUCCUAGCAAGCCAUCCAAGAGGUCUUUUAUCGAGGACC
UGCUGUUCAACAAGGUGACCCUGGCCGAUGCCGGCUUCAUCAAGCAGUAUGGCGAU
UGCCUGGGCGACAUCGCAGCCCGCGACCUGAUCUGUGCCCAGAAGUUUAAUGGCCU
GACCGUGCUGCCUCCACUGCUGACAGAUGAGAUGAUCGCCCAGUACACAUCUGCCC
UGCUGGCAGGAACCAUCACAAGCGGAUGGACCUUCGGCGCAGGAGCCGCCCUGCAG
AUCCCCUUUGCCAUGCAGAUGGCCUAUCGGUUCAACGGCAUCGGCGUGACCCAGAA
UGUGCUGUACGAGAACCAGAAGCUGAUCGCCAAUCAGUUUAACUCCGCCAUCGGCAA
GAUCCAGGACUCUCUGAGCAGCACAGCAAGCGCCCUGGGCAAGCUGCAGGAUGUGG
UGAAUCAGAACGCCCAGGCCCUGAAUACCCUGGUGAAGCAGCUGUCUAGCAACUUC
GGCGCCAUCUCCUCUGUGCUGAAUGAUAUCCUGAGCAGGCUGGACCCUCCAGAGGC
CGAAGUGCAGAUCGAUCGGCUGAUCACUGGUAGGCUGCAGAGUCUGCAGACAUAUG
UCACCCAGCAGCUGAUCAGAGCAGCCGAGAUUCGAGCUUCAGCUAACUUAGCUGCC
ACAAAGAUGAGUGAGUGCGUGCUGGGCCAGUCCAAGAGGGUGGAUUUCUGCGGAAA
AGGCUAUCAUCUCAUGUCCUUCCCCCAAAGCGCACCCCACGGGGUUGUCUUCCUGC
ACGUAACGUAUGUGCCCGCUCAGGAGAAGAACUUUACUACUGCCCCCGCCAUCUGC
CACGAUGGGAAAGCUCACUUCCCGAGAGAAGGAGUAUUCGUUUCCAAUGGGACGCA
UUGGUUCGUCACCCAGCGGAACUUUUACGAACCACAGAUCAUUACCACGGAUAAUAC
AUUUGUGAGCGGGAAUUGCGAUGUGGUGAUUGGAAUUGUAAAUAACACAGUGUACG
AUCCACUGCAGUCAGAAUUAGACUCAAUUAAGGAGGAGUUGGAUAAGAUUCAUAAAA
AUGGUUCAGGUGGAUCAGGUGAUAUAGAAAAACUCCUCAAUGAACAAGUAAAUAAGG
AGAUGCAAAGUUCUAACCUGUACAUGAGCAUGUCUUCUUGGUGUUACACCCAUAGCC
UCGAUGGAGCGGGAUUGUUCCUUUUUGACCACGCUGCGGAGGAGUAUGAGCAUGCU
AAAAAGCUGAUAAUAUUUCUCAACGAGAAUAAUGUUCCAGUGCAAUUGACAAGUAUA
UCCGCCCCUGAGCAUAAGUUUGAAGGGCUCACACAAAUUUUCCAAAAGGCAUACGAA
CACGAACAGCACAUUAGCGAGUCUAUUAACAACAUUGUUGAUCAUGCAAUCAAGUCC
AAAGAUCACGCCACGUUUAAUUUCCUCCAGUGGUAUGUAGCUGAGCAACAUGAGGAA
GAAGUGUUGUUUAAGGAUAUUCUUGAUAAAAUUGAACUUAUUGGAAAUGAGAACCAU
GGCCUCUAUCUUGCGGACCAAUACGUCAAGGGAAUUGCCAAGUCCCGCAAGAGU
SARS-CoV-2 Delta-linker-ferritin RNA sequence (SEQ ID NO: 2)
AUGCCCCUCCUUCUCCUUCUUCCCCUCCUUUGGGCUGGAGCGCUGGCCUCUCAGUG
UGUAAAUCUCCGUACCCGAACACAACUCCCCCCCGCCUAUACCAACAGCUUCACACG
CGGAGUUUACUACCCUGACAAGGUAUUCAGGUCCAGUGUACUCCAUUCAACACAGGA
UCUGUUCCUCCCUUUUUUCAGUAAUGUGACCUGGUUUCACGCUAUCCACGUAAGCG
GCACGAAUGGCACUAAGAGGUUCGAUAAUCCUGUACUGCCAUUUAAUGAUGGAGUU
UACUUCGCGAGCAUCGAGAAGAGCAACAUUAUCCGGGGGUGGAUCUUUGGAACAAC
UUUGGACAGUAAAACUCAAUCUCUCCUUAUUGUUAAUAACGCAACCAACGUAGUCAU
CAAGGUUUGCGAGUUCCAGUUUUGCAAUGACCCUUUUCUGGACGUUUAUUAUCAUA
AGAACAACAAGUCAUGGAUGGAAUCCGGCGUGUACUCUUCCGCAAAUAAUUGUACGU
UUGAGUAUGUCAGCCAGCCUUUCCUCAUGGACCUGGAAGGCAAACAGGGGAAUUUU
AAAAAUCUGAGGGAGUUCGUCUUCAAAAAUAUAGACGGGUACUUUAAAAUCUACUCC
AAGCACACGCCCAUCAAUUUAGUGAGGGACCUGCCCCAAGGCUUUUCUGCUCUUGA
ACCACUUGUGGACCUUCCCAUCGGCAUCAAUAUCACUCGCUUCCAGACACUCUUGGC
ACUUCAUAGAUCUUAUCUGACCCCAGGGGACAGCUCCUCAGGUUGGACCGCCGGCG
CUGCAGCUUACUACGUUGGAUAUUUGCAGCCACGCACGUUUCUUCUGAAAUACAAUG
AGAAUGGCACCAUUACCGACGCCGUAGAUUGCGCUCUCGAUCCUCUGAGCGAAACC
AAAUGUACAUUAAAGAGCUUUACGGUAGAAAAGGGGAUAUACCAAACCUCUAACUUU
CGGGUCCAGCCUACCGAAUCAAUCGUUCGGUUCCCAAACAUUACCAACUUAUGUCCA
UUUGGAGAAGUGUUCAAUGCCACGCGGUUCGCAAGCGUUUAUGCCUGGAACAGAAA
GCGCAUCUCUAAUUGCGUGGCCGAUUACUCAGUCCUCUACAAUUCAGCUAGUUUCA
GUACAUUUAAAUGUUACGGCGUGUCCCCCACUAAGUUGAAUGACCUUUGUUUUACUA
ACGUCUACGCUGACUCCUUCGUGAUCAGAGGCGAUGAGGUUCGGCAGAUCGCUCCU
GGGCAGACCGGUAAGAUCGCCGAUUACAACUACAAACUCCCAGACGACUUCACUGGC
UGUGUGAUUGCCUGGAACAGUAACAAUUUAGACUCCAAAGUAGGGGGUAACUACAAC
UAUCGAUAUAGACUUUUCCGGAAAAGUAACUUGAAACCUUUUGAAAGAGAUAUAUCC
ACUGAAAUCUAUCAGGCCGGAAGUAAGCCCUGUAAUGGAGUCGAGGGCUUCAAUUG
CUACUUCCCAUUACAGAGCUAUGGCUUCCAGCCUACGAACGGCGUCGGAUAUCAGC
CUUACCGCGUGGUGGUCUUGUCUUUCGAACUGUUACAUGCACCUGCGACAGUGUGC
GGGCCUAAGAAAUCCACCAACCUGGUGAAGAACAAAUGCGUGAACUUUAAUUUCAAC
GGACUGACUGGCACAGGCGUUCUGACUGAGAGCAACAAGAAAUUUCUGCCCUUCCA
GCAAUUUGGCCGUGACAUUGCCGACACCACGGACGCUGUACGCGAUCCGCAAACCC
UGGAGAUCCUGGACAUCACCCCAUGCUCUUUUGGCGGGGUCAGCGUUAUUACACCU
GGGACCAACACAAGCAACCAGGUUGCUGUCUUAUACCAGGGGGUUAAUUGCACCGA
AGUGCCUGUCGCAAUCCAUGCUGACCAACUUACCCCCACUUGGCGCGUUUAUUCUA
CCGGCUCCAACGUAUUCCAAACCAGGGCUGGCUGUCUCAUCGGAGCAGAGCAUGUG
AACAACUCUUACGAAUGCGAUAUACCAAUUGGCGCGGGAAUCUGUGCCUCCUAUCAG
ACACAAACAAACAGUCGCGGAAGCGCGAGCAGUGUGGCAUCACAGAGUAUCAUCGCA
UACACCAUGAGCCUUGGCGCAGAGAACAGCGUGGCCUACUCUAAUAACAGUAUUGCC
AUACCUACCAACUUCACCAUUAGCGUGACGACCGAGAUCCUGCCCGUGUCUAUGACC
AAGACAUCUGUGGAUUGCACAAUGUAUAUCUGCGGAGACAGCACUGAAUGCUCCAAU
CUGCUUUUGCAGUACGGGUCUUUCUGUACCCAGCUGAAUCGAGCACUGACAGGAAU
CGCUGUGGAACAGGAUAAGAACACCCAGGAGGUGUUCGCCCAGGUUAAGCAGAUAU
ACAAGACCCCUCCUAUAAAAGACUUCGGUGGCUUUAACUUUUCUCAGAUUCUCCCCG
ACCCUUCUAAGCCUUCUAAACGAUCCUUCAUCGAAGACCUUCUCUUCAACAAAGUUA
CUUUGGCUGAUGCCGGGUUCAUCAAGCAGUAUGGUGACUGUCUUGGCGAUAUCGCC
GCCAGAGAUUUAAUAUGUGCCCAGAAGUUCAACGGUUUAACAGUCCUUCCUCCUCUG
CUGACAGACGAGAUGAUUGCUCAGUACACUAGUGCUCUGCUCGCCGGGACUAUUAC
UUCAGGCUGGACCUUUGGCGCUGGCGCCGCCCUGCAGAUUCCCUUUGCUAUGCAAA
UGGCAUACCGGUUCAACGGUAUAGGUGUGACUCAGAAUGUGCUGUAUGAAAAUCAG
AAACUCAUUGCAAAUCAGUUUAACAGUGCUAUUGGGAAGAUACAGGACUCCCUCUCU
UCCACCGCGUCCGCUUUAGGUAAGCUUCAAAACGUGGUAAAUCAAAAUGCCCAGGCU
UUAAAUACCUUGGUUAAGCAACUGAGUUCAAAUUUCGGAGCAAUCUCUAGCGUGCUU
AACGAUAUCCUCUCUAGGCUGGAUCCACCCGAGGCCGAAGUGCAGAUCGAUCGGCU
GAUCACUGGUAGGCUGCAGAGUCUGCAGACAUAUGUCACCCAGCAGCUGAUCAGAG
CAGCCGAGAUUCGAGCUUCAGCUAACUUAGCUGCCACAAAGAUGAGUGAGUGCGUG
CUGGGCCAGUCCAAGAGGGUGGAUUUCUGCGGAAAAGGCUAUCAUCUCAUGUCCUU
CCCCCAAAGCGCACCCCACGGGGUUGUCUUCCUGCACGUAACGUAUGUGCCCGCUC
AGGAGAAGAACUUUACUACUGCCCCCGCCAUCUGCCACGAUGGGAAAGCUCACUUCC
CGAGAGAAGGAGUAUUCGUUUCCAAUGGGACGCAUUGGUUCGUCACCCAGCGGAAC
UUUUACGAACCACAGAUCAUUACCACGGAUAAUACAUUUGUGAGCGGGAAUUGCGAU
GUGGUGAUUGGAAUUGUAAAUAACACAGUGUACGAUCCACUGCAGUCAGAAUUAGAC
UCAAUUAAGGAGGAGUUGGAUAAGAUUCAUAAAAAUGGUUCAGGUGGAUCAGGUGAU
AUAGAAAAACUCCUCAAUGAACAAGUAAAUAAGGAGAUGCAAAGUUCUAACCUGUACA
UGAGCAUGUCUUCUUGGUGUUACACCCAUAGCCUCGAUGGAGCGGGAUUGUUCCUU
UUUGACCACGCUGCGGAGGAGUAUGAGCAUGCUAAAAAGCUGAUAAUAUUUCUCAAC
GAGAAUAAUGUUCCAGUGCAAUUGACAAGUAUAUCCGCCCCUGAGCAUAAGUUUGAA
GGGCUCACACAAAUUUUCCAAAAGGCAUACGAACACGAACAGCACAUUAGCGAGUCU
AUUAACAACAUUGUUGAUCAUGCAAUCAAGUCCAAAGAUCACGCCACGUUUAAUUUC
CUCCAGUGGUAUGUAGCUGAGCAACAUGAGGAAGAAGUGUUGUUUAAGGAUAUUCU
UGAUAAAAUUGAACUUAUUGGAAAUGAGAACCAUGGCCUCUAUCUUGCGGACCAAUA
CGUCAAGGGAAUUGCCAAGUCCCGCAAGAGU
SARS-CoV-2 Omicron BA.4/5-linker-ferritin RNA sequence (SEQ ID NO: 3)
AUGCCCCUCCUUCUCCUUCUUCCCCUCCUUUGGGCUGGAGCGCUGGCCUCUCAGUG
UGUAAAUCUCAUUACCCGCACGCAGCUACCACCUGCCUAUACAAACUCCUUCACCCG
CGGUGUCUAUUACCCCGACAAGGUUUUUCGAUCCUCAGUUCUUCAUUCCACCCAGG
AUCUUUUCCUUCCGUUCUUCUCCAACGUCACCUGGUUUCAUGCUAUUCAUGUGUCA
GGGACCAACGGAACUAAAAGGUUCGACAAUCCCGUACUGCCUUUCAACGACGGGGU
GUAUUUCGCAUCAACAGAAAAAAGCAACAUUAUCAGAGGCUGGAUUUUUGGCACUAC
ACUCGACUCAAAGACCCAAAGUUUGCUUAUUGUUAAUAACGCAACGAAUGUCGUAAU
UAAAGUCUGUGAAUUCCAGUUUUGUAACGACCCAUUUCUGGAUGUAUACUAUCAUAA
GAACAAUAAGUCCUGGAUGGAGUCAGAGUUCAGAGUCUACUCAUCAGCAAACAAUUG
CACUUUUGAGUACGUAUCUCAGCCAUUUCUGAUGGACCUGGAAGGCAAGCAAGGGA
ACUUCAAAAAUCUCCGAGAGUUUGUUUUCAAAAACAUAGAUGGUUACUUCAAGAUCU
AUUCUAAACACACCCCGAUUAAUCUUGGCCGGGACCUGCCCCAGGGUUUCUCUGCC
CUCGAGCCCCUUGUAGACUUGCCCAUUGGGAUAAAUAUUACUAGGUUUCAGACACU
GCUUGCGCUGCACAGGUCAUACCUCACUCCUGGGGAUUCAUCAAGCGGCUGGACUG
CAGGUGCCGCUGCAUACUACGUGGGAUACCUCCAACCCCGAACUUUUUUGCUCAAG
UACAAUGAGAAUGGGACGAUAACUGAUGCCGUAGACUGCGCACUCGAUCCUCUGUC
UGAGACGAAGUGCACACUGAAGAGUUUCACGGUGGAAAAGGGUAUAUAUCAAACAUC
CAAUUUCCGAGUCCAGCCCACUGAAAGUAUUGUUAGGUUCCCGAACAUCACAAAUCU
CUGUCCAUUUGACGAGGUAUUUAACGCGACACGCUUUGCUUCCGUGUAUGCGUGGA
AUAGAAAGCGCAUUAGCAACUGUGUCGCGGAUUAUUCAGUCUUGUACAACUUUGCG
CCUUUUUUCGCAUUUAAAUGCUACGGGGUCAGCCCCACAAAGCUGAAUGAUCUCUG
CUUCACAAAUGUAUAUGCAGAUAGCUUUGUCAUCCGAGGUAACGAAGUUUCUCAGAU
UGCUCCCGGCCAAACGGGAAACAUAGCUGACUAUAACUAUAAGCUCCCAGACGAUUU
UACGGGUUGUGUCAUUGCAUGGAACUCCAAUAAAUUGGAUUCAAAGGUAGGUGGUA
AUUAUAACUACAGGUAUAGGCUGUUCAGGAAGUCAAACCUGAAACCGUUUGAACGAG
AUAUCUCAACUGAGAUUUAUCAAGCGGGCAAUAAACCCUGCAACGGAGUCGCGGGU
GUUAAUUGCUACUUCCCAUUGCAGAGUUACGGCUUUCGGCCGACUUAUGGCGUCGG
ACAUCAACCUUACAGGGUAGUUGUUCUGUCUUUCGAACUCCUGCAUGCUCCGGCAA
CGGUGUGUGGUCCCAAGAAAAGUACUAACCUUGUCAAAAAUAAGUGUGUGAACUUCA
AUUUUAACGGUCUUACAGGUACUGGAGUAUUGACUGAGUCAAAUAAGAAGUUUCUUC
CAUUCCAACAAUUCGGCCGCGAUAUAGCUGAUACAACGGAUGCCGUCCGAGACCCUC
AGACUCUGGAGAUCCUGGACAUAACUCCAUGUUCUUUUGGAGGCGUUAGUGUGAUU
ACCCCAGGCACUAAUACCUCAAACCAAGUAGCCGUCCUGUAUCAAGGAGUCAAUUGU
ACUGAGGUCCCUGUGGCCAUCCAUGCGGACCAACUGACACCCACAUGGCGCGUAUA
UAGCACAGGAUCAAACGUAUUCCAGACUCGCGCGGGUUGUCUGAUCGGGGCGGAAU
ACGUAAACAAUAGCUAUGAGUGUGAUAUACCUAUUGGAGCUGGGAUUUGCGCAAGC
UAUCAAACACAAACGAAAUCCCACGGAUCGGCAUCAUCAGUUGCAUCUCAAUCUAUU
AUCGCUUAUACAAUGUCUCUUGGCGCUGAGAACUCCGUUGCUUACAGCAAUAAUAGC
AUUGCCAUACCGACGAACUUUACUAUUAGCGUAACGACUGAGAUACUCCCGGUGUCU
AUGACAAAAACUAGCGUUGAUUGUACAAUGUACAUUUGUGGGGAUAGUACAGAGUGC
UCAAACUUGCUUCUCCAGUACGGGUCAUUCUGUACCCAACUUAAACGCGCAUUGACU
GGUAUUGCCGUCGAGCAAGACAAAAACACACAAGAGGUUUUCGCUCAAGUAAAACAA
AUUUACAAAACGCCCCCUAUAAAAUAUUUUGGCGGUUUCAACUUUUCUCAAAUUCUG
CCCGAUCCUUCUAAGCCUAGUAAGCGAAGCUUCAUUGAAGACCUGCUUUUCAACAAA
GUAACCUUGGCUGACGCGGGUUUCAUAAAACAGUAUGGAGACUGUCUCGGGGACAU
AGCCGCAAGGGACUUGAUUUGUGCCCAAAAAUUCAACGGACUGACAGUUCUUCCUCC
ACUUUUGACUGAUGAGAUGAUAGCACAAUACACUUCUGCUUUGCUGGCCGGUACAAU
AACGAGUGGGUGGACCUUCGGGGCUGGGGCUGCCCUCCAAAUACCCUUCGCUAUGC
AAAUGGCAUAUCGAUUUAAUGGCAUCGGGGUUACACAGAACGUCCUGUAUGAAAACC
AAAAGCUCAUUGCCAACCAGUUUAACUCAGCAAUCGGAAAAAUUCAGGACAGCCUGA
GUAGUACCGCCAGCGCCUUGGGAAAGUUGCAGGAUGUCGUCAACCAUAACGCCCAA
GCAUUGAAUACGUUGGUCAAACAGCUCUCCUCUAAGUUCGGUGCUAUAUCCUCAGU
CCUCAACGAUAUCCUGAGCAGGCUCGAUCCCCCUGAGGCCGAAGUGCAGAUCGAUC
GGCUGAUCACUGGUAGGCUGCAGAGUCUGCAGACAUAUGUCACCCAGCAGCUGAUC
AGAGCAGCCGAGAUUCGAGCUUCAGCUAACUUAGCUGCCACAAAGAUGAGUGAGUG
CGUGCUGGGCCAGUCCAAGAGGGUGGAUUUCUGCGGAAAAGGCUAUCAUCUCAUGU
CCUUCCCCCAAAGCGCACCCCACGGGGUUGUCUUCCUGCACGUAACGUAUGUGCCC
GCUCAGGAGAAGAACUUUACUACUGCCCCCGCCAUCUGCCACGAUGGGAAAGCUCA
CUUCCCGAGAGAAGGAGUAUUCGUUUCCAAUGGGACGCAUUGGUUCGUCACCCAGC
GGAACUUUUACGAACCACAGAUCAUUACCACGGAUAAUACAUUUGUGAGCGGGAAUU
GCGAUGUGGUGAUUGGAAUUGUAAAUAACACAGUGUACGAUCCACUGCAGUCAGAAU
UAGACUCAAUUAAGGAGGAGUUGGAUAAGAUUCAUAAAAAUGGUUCAGGUGGAUCAG
GUGAUAUAGAAAAACUCCUCAAUGAACAAGUAAAUAAGGAGAUGCAAAGUUCUAACCU
GUACAUGAGCAUGUCUUCUUGGUGUUACACCCAUAGCCUCGAUGGAGCGGGAUUGU
UCCUUUUUGACCACGCUGCGGAGGAGUAUGAGCAUGCUAAAAAGCUGAUAAUAUUUC
UCAACGAGAAUAAUGUUCCAGUGCAAUUGACAAGUAUAUCCGCCCCUGAGCAUAAGU
UUGAAGGGCUCACACAAAUUUUCCAAAAGGCAUACGAACACGAACAGCACAUUAGCG
AGUCUAUUAACAACAUUGUUGAUCAUGCAAUCAAGUCCAAAGAUCACGCCACGUUUA
AUUUCCUCCAGUGGUAUGUAGCUGAGCAACAUGAGGAAGAAGUGUUGUUUAAGGAU
AUUCUUGAUAAAAUUGAACUUAUUGGAAAUGAGAACCAUGGCCUCUAUCUUGCGGAC
CAAUACGUCAAGGGAAUUGCCAAGUCCCGCAAGAGU
Linker amino acid sequence: GSGGSG (SEQ ID NO: 4).
Linker RNA sequence: GGUUCAGGUGGAUCAGGU (SEQ ID NO: 5)
Ferritin subunit, RNA sequence (SEQ ID NO: 6):
GAUAUAGAAAAACUCCUCAAUGAACAAGUAAAUAAGGAGAUGCAAAGUUCUAACCUGU
ACAUGAGCAUGUCUUCUUGGUGUUACACCCAUAGCCUCGAUGGAGCGGGAUUGUUC
CUUUUUGACCACGCUGCGGAGGAGUAUGAGCAUGCUAAAAAGCUGAUAAUAUUUCUC
AACGAGAAUAAUGUUCCAGUGCAAUUGACAAGUAUAUCCGCCCCUGAGCAUAAGUUU
GAAGGGCUCACACAAAUUUUCCAAAAGGCAUACGAACACGAACAGCACAUUAGCGAG
UCUAUUAACAACAUUGUUGAUCAUGCAAUCAAGUCCAAAGAUCACGCCACGUUUAAU
UUCCUCCAGUGGUAUGUAGCUGAGCAACAUGAGGAAGAAGUGUUGUUUAAGGAUAU
UCUUGAUAAAAUUGAACUUAUUGGAAAUGAGAACCAUGGCCUCUAUCUUGCGGACCA
AUACGUCAAGGGAAUUGCCAAGUCCCGCAAGAGU
Ferritin subunit, protein sequence (SEQ ID NO: 7)
DIEKLLNEQVNKEMQSSNLYMSMSSWCYTHSLDGAGLFLFDHAAEEYEHAKKLIIFLNENNV
PVQLTSISAPEHKFEGLTQIFQKAYEHEQHISESINNIVDHAIKSKDHATFNFLQWYVAEQHE
EEVLFKDILDKIELIGNENHGLYLADQYVKGIAKSRKS
Leader sequence: MPLLLLLPLLWAGALA (SEQ ID NO: 8).
mRNA construct sequence encoding SARS-CoV-2 Wuhan D614G Spike-ferritin
fusion protein (SEQ ID NO: 9)
AGGAUUGUGCUGCAUCAAGCUUGCCGCCACCAUGCCCCUCCUUCUCCUUCUUCCCC
UCCUUUGGGCUGGAGCGCUGGCCUCUCAGUGUGUAAAUCUCACCACAAGAACCCAG
CUGCCCCCUGCCUAUACCAAUUCCUUCACACGGGGCGUGUACUAUCCCGACAAGGU
GUUUAGAUCUAGCGUGCUGCACUCCACACAGGAUCUGUUUCUGCCUUUCUUUUCUA
ACGUGACCUGGUUCCACGCCAUCCACGUGAGCGGCACCAAUGGCACAAAGAGGUUC
GACAAUCCAGUGCUGCCCUUUAACGAUGGCGUGUACUUCGCCUCCACCGAGAAGUC
UAACAUCAUCCGCGGCUGGAUCUUUGGCACCACACUGGACAGCAAGACACAGUCCCU
GCUGAUCGUGAACAAUGCCACCAACGUGGUCAUCAAGGUGUGCGAGUUCCAGUUUU
GUAAUGAUCCAUUCCUGGGCGUGUACUAUCACAAGAACAAUAAGUCUUGGAUGGAGA
GCGAGUUUCGGGUGUAUUCCUCUGCCAACAAUUGCACAUUUGAGUACGUGUCCCAG
CCCUUCCUGAUGGACCUGGAGGGCAAGCAGGGCAAUUUCAAGAACCUGCGGGAGUU
CGUGUUUAAGAAUAUCGAUGGCUACUUCAAGAUCUACUCCAAGCACACCCCCAUCAA
CCUGGUGCGGGACCUGCCACAGGGCUUCUCUGCCCUGGAGCCACUGGUGGAUCUG
CCCAUCGGCAUCAACAUCACCCGGUUUCAGACACUGCUGGCCCUGCACAGAAGCUAC
CUGACACCAGGCGACAGCUCCUCUGGAUGGACCGCAGGAGCAGCAGCCUACUAUGU
GGGCUAUCUGCAGCCCCGGACCUUCCUGCUGAAGUACAACGAGAAUGGCACCAUCA
CAGACGCCGUGGAUUGCGCCCUGGAUCCCCUGUCUGAGACAAAGUGUACACUGAAG
AGUUUUACCGUGGAGAAGGGCAUCUAUCAGACAAGCAAUUUCAGGGUGCAGCCUAC
CGAGUCCAUCGUGCGCUUUCCCAAUAUCACAAACCUGUGCCCUUUUGGCGAGGUGU
UCAACGCCACCAGAUUCGCCAGCGUGUACGCCUGGAAUAGGAAGCGCAUCUCCAAC
UGCGUGGCCGACUAUUCUGUGCUGUACAACAGCGCCUCCUUCUCUACCUUUAAGUG
CUAUGGCGUGAGCCCCACAAAGCUGAAUGACCUGUGCUUUACCAACGUGUACGCCG
AUUCCUUCGUGAUCAGGGGCGACGAGGUGCGCCAGAUCGCACCAGGACAGACAGGC
AAGAUCGCAGACUACAAUUAUAAGCUGCCUGACGAUUUCACCGGCUGCGUGAUCGC
CUGGAACUCUAACAAUCUGGAUAGCAAAGUGGGCGGCAACUACAAUUAUCUGUACCG
GCUGUUUAGAAAGUCUAAUCUGAAGCCAUUCGAGCGGGACAUCUCCACAGAGAUCUA
CCAGGCCGGCUCUACCCCCUGCAAUGGCGUGGAGGGCUUUAACUGUUAUUUCCCUC
UGCAGAGCUACGGCUUCCAGCCAACCAACGGCGUGGGCUAUCAGCCCUACAGAGUG
GUGGUGCUGUCUUUUGAGCUGCUGCACGCACCUGCAACAGUGUGCGGACCAAAAAA
GAGCACCAAUCUGGUGAAGAACAAGUGCGUGAACUUCAACUUCAACGGCCUGACCG
GAACAGGCGUGCUGACCGAGUCCAACAAGAAGUUCCUGCCUUUUCAGCAGUUCGGC
AGGGACAUCGCAGAUACCACAGACGCCGUGCGCGACCCUCAGACCCUGGAGAUCCU
GGAUAUCACACCAUGCUCCUUCGGCGGCGUGUCUGUGAUCACACCAGGCACCAAUA
CAAGCAACCAGGUGGCCGUGCUGUAUCAGGGCGUGAAUUGUACCGAGGUGCCAGUG
GCAAUCCACGCAGAUCAGCUGACCCCUACAUGGCGGGUGUACUCUACCGGCAGCAA
CGUGUUCCAGACAAGAGCCGGAUGCCUGAUCGGAGCAGAGCACGUGAACAAUAGCU
AUGAGUGCGACAUCCCUAUCGGCGCCGGCAUCUGUGCCUCCUACCAGACCCAGACA
AACUCCCCAGGGUCUGCAUCAUCUGUGGCAAGCCAGUCCAUCAUCGCCUAUACCAU
GAGCCUGGGCGCCGAGAAUUCCGUGGCCUACUCCAACAAUUCUAUCGCCAUCCCUA
CCAACUUCACAAUCUCCGUGACCACAGAGAUCCUGCCAGUGAGCAUGACCAAGACAU
CCGUGGACUGCACAAUGUAUAUCUGUGGCGAUUCCACCGAGUGCUCUAACCUGCUG
CUGCAGUACGGCAGCUUUUGUACCCAGCUGAAUCGCGCCCUGACAGGAAUCGCAGU
GGAGCAGGAUAAGAACACACAGGAGGUGUUCGCCCAGGUGAAGCAGAUCUACAAGA
CCCCACCCAUCAAGGACUUUGGCGGCUUCAAUUUUUCCCAGAUCCUGCCCGAUCCU
AGCAAGCCAUCCAAGAGGUCUUUUAUCGAGGACCUGCUGUUCAACAAGGUGACCCU
GGCCGAUGCCGGCUUCAUCAAGCAGUAUGGCGAUUGCCUGGGCGACAUCGCAGCCC
GCGACCUGAUCUGUGCCCAGAAGUUUAAUGGCCUGACCGUGCUGCCUCCACUGCUG
ACAGAUGAGAUGAUCGCCCAGUACACAUCUGCCCUGCUGGCAGGAACCAUCACAAGC
GGAUGGACCUUCGGCGCAGGAGCCGCCCUGCAGAUCCCCUUUGCCAUGCAGAUGGC
CUAUCGGUUCAACGGCAUCGGCGUGACCCAGAAUGUGCUGUACGAGAACCAGAAGC
UGAUCGCCAAUCAGUUUAACUCCGCCAUCGGCAAGAUCCAGGACUCUCUGAGCAGCA
CAGCAAGCGCCCUGGGCAAGCUGCAGGAUGUGGUGAAUCAGAACGCCCAGGCCCUG
AAUACCCUGGUGAAGCAGCUGUCUAGCAACUUCGGCGCCAUCUCCUCUGUGCUGAA
UGAUAUCCUGAGCAGGCUGGACCCUCCAGAGGCCGAAGUGCAGAUCGAUCGGCUGA
UCACUGGUAGGCUGCAGAGUCUGCAGACAUAUGUCACCCAGCAGCUGAUCAGAGCA
GCCGAGAUUCGAGCUUCAGCUAACUUAGCUGCCACAAAGAUGAGUGAGUGCGUGCU
GGGCCAGUCCAAGAGGGUGGAUUUCUGCGGAAAAGGCUAUCAUCUCAUGUCCUUCC
CCCAAAGCGCACCCCACGGGGUUGUCUUCCUGCACGUAACGUAUGUGCCCGCUCAG
GAGAAGAACUUUACUACUGCCCCCGCCAUCUGCCACGAUGGGAAAGCUCACUUCCC
GAGAGAAGGAGUAUUCGUUUCCAAUGGGACGCAUUGGUUCGUCACCCAGCGGAACU
UUUACGAACCACAGAUCAUUACCACGGAUAAUACAUUUGUGAGCGGGAAUUGCGAUG
UGGUGAUUGGAAUUGUAAAUAACACAGUGUACGAUCCACUGCAGUCAGAAUUAGACU
CAAUUAAGGAGGAGUUGGAUAAGAUUCAUAAAAAUGGUUCAGGUGGAUCAGGUGAUA
UAGAAAAACUCCUCAAUGAACAAGUAAAUAAGGAGAUGCAAAGUUCUAACCUGUACAU
GAGCAUGUCUUCUUGGUGUUACACCCAUAGCCUCGAUGGAGCGGGAUUGUUCCUUU
UUGACCACGCUGCGGAGGAGUAUGAGCAUGCUAAAAAGCUGAUAAUAUUUCUCAACG
AGAAUAAUGUUCCAGUGCAAUUGACAAGUAUAUCCGCCCCUGAGCAUAAGUUUGAAG
GGCUCACACAAAUUUUCCAAAAGGCAUACGAACACGAACAGCACAUUAGCGAGUCUA
UUAACAACAUUGUUGAUCAUGCAAUCAAGUCCAAAGAUCACGCCACGUUUAAUUUCC
UCCAGUGGUAUGUAGCUGAGCAACAUGAGGAAGAAGUGUUGUUUAAGGAUAUUCUU
GAUAAAAUUGAACUUAUUGGAAAUGAGAACCAUGGCCUCUAUCUUGCGGACCAAUAC
GUCAAGGGAAUUGCCAAGUCCCGCAAGAGUUGAUGAUAAUAGGACUAGUGGAUCCAA
CUGGAGACUGGGUGAAAGUGACUACCAGAAAGUGAGGAAGCCUAAAUAAACCUAGCG
UACGUAAAAAAUGGAAAGAACCUAGCGUACGAAAAAAAAAAAAAAAAAAAAAAAAAAAA
AAAAAAAAAAAAAAAAAAAAAAAAAAAAAAAAAAAAAAAAAAAAAAAAA
mRNA construct sequence encoding SARS-CoV-2 Delta Spike-ferritin
fusion protein (SEQ ID NO: 10)
AGGAUUGUGCUGCAUCAAGCUUGCCGCCACCAUGCCCCUCCUUCUCCUUCUUCCCC
UCCUUUGGGCUGGAGCGCUGGCCUCUCAGUGUGUAAAUCUCCGUACCCGAACACAA
CUCCCCCCCGCCUAUACCAACAGCUUCACACGCGGAGUUUACUACCCUGACAAGGUA
UUCAGGUCCAGUGUACUCCAUUCAACACAGGAUCUGUUCCUCCCUUUUUUCAGUAAU
GUGACCUGGUUUCACGCUAUCCACGUAAGCGGCACGAAUGGCACUAAGAGGUUCGA
UAAUCCUGUACUGCCAUUUAAUGAUGGAGUUUACUUCGCGAGCAUCGAGAAAAGCAA
CAUUAUCCGGGGGUGGAUCUUUGGAACAACUUUGGACAGUAAAACUCAAUCUCUCCU
UAUUGUUAAUAACGCAACCAACGUAGUCAUCAAGGUUUGCGAGUUCCAGUUUUGCAA
UGACCCUUUUCUGGACGUUUAUUAUCAUAAGAACAACAAGUCAUGGAUGGAAUCCGG
CGUGUACUCUUCCGCAAAUAAUUGUACGUUUGAGUAUGUCAGCCAGCCUUUCCUCA
UGGACCUGGAAGGCAAACAGGGGAAUUUUAAAAAUCUGAGGGAGUUCGUCUUCAAAA
AUAUAGACGGGUACUUUAAAAUCUACUCCAAGCACACGCCCAUCAAUUUAGUGAGGG
ACCUGCCCCAAGGCUUUUCUGCUCUUGAACCACUUGUGGACCUUCCCAUCGGCAUC
AAUAUCACUCGCUUCCAGACACUCUUGGCACUUCAUAGAUCUUAUCUGACCCCAGGG
GACAGCUCCUCAGGUUGGACCGCCGGCGCUGCAGCUUACUACGUUGGAUAUUUGCA
GCCACGCACGUUUCUUCUGAAAUACAAUGAGAAUGGCACCAUUACCGACGCCGUAGA
UUGCGCUCUCGAUCCUCUGAGCGAAACCAAAUGUACAUUAAAGAGCUUUACGGUAGA
AAAGGGGAUAUACCAAACCUCUAACUUUCGGGUCCAGCCUACCGAAUCAAUCGUUCG
GUUCCCAAACAUUACCAACUUAUGUCCAUUUGGAGAAGUGUUCAAUGCCACGCGGUU
CGCAAGCGUUUAUGCCUGGAACAGAAAGCGCAUCUCUAAUUGCGUGGCCGAUUACU
CAGUCCUCUACAAUUCAGCUAGUUUCAGUACAUUUAAAUGUUACGGCGUGUCCCCCA
CUAAGUUGAAUGACCUUUGUUUUACUAACGUCUACGCUGACUCCUUCGUGAUCAGA
GGCGAUGAGGUUCGGCAGAUCGCUCCUGGGCAGACCGGUAAGAUCGCCGAUUACAA
CUACAAACUCCCAGACGACUUCACUGGCUGUGUGAUUGCCUGGAACAGUAACAAUUU
AGACUCCAAAGUAGGGGGUAACUACAACUAUCGAUAUAGACUUUUCCGGAAAAGUAA
CUUGAAACCUUUUGAAAGAGAUAUAUCCACUGAAAUCUAUCAGGCCGGAAGUAAGCC
CUGUAAUGGAGUCGAGGGCUUCAAUUGCUACUUCCCAUUACAGAGCUAUGGCUUCC
AGCCUACGAACGGCGUCGGAUAUCAGCCUUACCGCGUGGUGGUCUUGUCUUUCGAA
CUGUUACAUGCACCUGCGACAGUGUGCGGGCCUAAGAAAUCCACCAACCUGGUGAA
GAACAAAUGCGUGAACUUUAAUUUCAACGGACUGACUGGCACAGGCGUUCUGACUGA
GAGCAACAAGAAAUUUCUGCCCUUCCAGCAAUUUGGCCGUGACAUUGCCGACACCAC
GGACGCUGUACGCGAUCCGCAAACCCUGGAGAUCCUGGACAUCACCCCAUGCUCUU
UUGGCGGGGUCAGCGUUAUUACACCUGGGACCAACACAAGCAACCAGGUUGCUGUC
UUAUACCAGGGGGUUAAUUGCACCGAAGUGCCUGUCGCAAUCCAUGCUGACCAACU
UACCCCCACUUGGCGCGUUUAUUCUACCGGCUCCAACGUAUUCCAAACCAGGGCUG
GCUGUCUCAUCGGAGCAGAGCAUGUGAACAACUCUUACGAAUGCGAUAUACCAAUUG
GCGCGGGAAUCUGUGCCUCCUAUCAGACACAAACAAACAGUCGCGGAAGCGCGAGC
AGUGUGGCAUCACAGAGUAUCAUCGCAUACACCAUGAGCCUUGGCGCAGAGAACAG
CGUGGCCUACUCUAAUAACAGUAUUGCCAUACCUACCAACUUCACCAUUAGCGUGAC
GACCGAGAUCCUGCCCGUGUCUAUGACCAAGACAUCUGUGGAUUGCACAAUGUAUA
UCUGCGGAGACAGCACUGAAUGCUCCAAUCUGCUUUUGCAGUACGGGUCUUUCUGU
ACCCAGCUGAAUCGAGCACUGACAGGAAUCGCUGUGGAACAGGAUAAGAACACCCAG
GAGGUGUUCGCCCAGGUUAAGCAGAUAUACAAGACCCCUCCUAUAAAAGACUUCGGU
GGCUUUAACUUUUCUCAGAUUCUCCCCGACCCUUCUAAGCCUUCUAAACGAUCCUUC
AUCGAAGACCUUCUCUUCAACAAAGUUACUUUGGCUGAUGCCGGGUUCAUCAAGCAG
UAUGGUGACUGUCUUGGCGAUAUCGCCGCCAGAGAUUUAAUAUGUGCCCAGAAGUU
CAACGGUUUAACAGUCCUUCCUCCUCUGCUGACAGACGAGAUGAUUGCUCAGUACAC
UAGUGCUCUGCUCGCCGGGACUAUUACUUCAGGCUGGACCUUUGGCGCUGGCGCC
GCCCUGCAGAUUCCCUUUGCUAUGCAAAUGGCAUACCGGUUCAACGGUAUAGGUGU
GACUCAGAAUGUGCUGUAUGAAAAUCAGAAACUCAUUGCAAAUCAGUUUAACAGUGC
UAUUGGGAAGAUACAGGACUCCCUCUCUUCCACCGCGUCCGCUUUAGGUAAGCUUC
AAAACGUGGUAAAUCAAAAUGCCCAGGCUUUAAAUACCUUGGUUAAGCAACUGAGUU
CAAAUUUCGGAGCAAUCUCUAGCGUGCUUAACGAUAUCCUCUCUAGGCUGGAUCCAC
CCGAGGCCGAAGUGCAGAUCGAUCGGCUGAUCACUGGUAGGCUGCAGAGUCUGCAG
ACAUAUGUCACCCAGCAGCUGAUCAGAGCAGCCGAGAUUCGAGCUUCAGCUAACUUA
GCUGCCACAAAGAUGAGUGAGUGCGUGCUGGGCCAGUCCAAGAGGGUGGAUUUCUG
CGGAAAAGGCUAUCAUCUCAUGUCCUUCCCCCAAAGCGCACCCCACGGGGUUGUCU
UCCUGCACGUAACGUAUGUGCCCGCUCAGGAGAAGAACUUUACUACUGCCCCCGCC
AUCUGCCACGAUGGGAAAGCUCACUUCCCGAGAGAAGGAGUAUUCGUUUCCAAUGG
GACGCAUUGGUUCGUCACCCAGCGGAACUUUUACGAACCACAGAUCAUUACCACGGA
UAAUACAUUUGUGAGCGGGAAUUGCGAUGUGGUGAUUGGAAUUGUAAAUAACACAG
UGUACGAUCCACUGCAGUCAGAAUUAGACUCAAUUAAGGAGGAGUUGGAUAAGAUUC
AUAAAAAUGGUUCAGGUGGAUCAGGUGAUAUAGAAAAACUCCUCAAUGAACAAGUAA
AUAAGGAGAUGCAAAGUUCUAACCUGUACAUGAGCAUGUCUUCUUGGUGUUACACCC
AUAGCCUCGAUGGAGCGGGAUUGUUCCUUUUUGACCACGCUGCGGAGGAGUAUGAG
CAUGCUAAAAAGCUGAUAAUAUUUCUCAACGAGAAUAAUGUUCCAGUGCAAUUGACA
AGUAUAUCCGCCCCUGAGCAUAAGUUUGAAGGGCUCACACAAAUUUUCCAAAAGGCA
UACGAACACGAACAGCACAUUAGCGAGUCUAUUAACAACAUUGUUGAUCAUGCAAUC
AAGUCCAAAGAUCACGCCACGUUUAAUUUCCUCCAGUGGUAUGUAGCUGAGCAACAU
GAGGAAGAAGUGUUGUUUAAGGAUAUUCUUGAUAAAAUUGAACUUAUUGGAAAUGAG
AACCAUGGCCUCUAUCUUGCGGACCAAUACGUCAAGGGAAUUGCCAAGUCCCGCAAG
AGUUGAUGAUAAUAGGACUAGUGGAUCCAACUGGAGACUGGGUGAAAGUGACUACC
AGAAAGUGAGGAAGCCUAAAUAAACCUAGCGUACGUAAAAAAUGGAAAGAACCUAGC
GUACGAAAAAAAAAAAAAAAAAAAAAAAAAAAAAAAAAAAAAAAAAAAAAAAAAAAAAAAA
AAAAAAAAAAAAAAAAAAAAA
mRNA construct sequence encoding SARS-CoV-2 Omicron BA.4/5 Spike-
ferritin fusion protein (SEQ ID NO: 11)
AGGAUUGUGCUGCAUCAAGCUUGCCGCCACCAUGCCCCUCCUUCUCCUUCUUCCCC
UCCUUUGGGCUGGAGCGCUGGCCUCUCAGUGUGUAAAUCUCAUUACCCGCACGCAG
CUACCACCUGCCUAUACAAACUCCUUCACCCGCGGUGUCUAUUACCCCGACAAGGUU
UUUCGAUCCUCAGUUCUUCAUUCCACCCAGGAUCUUUUCCUUCCGUUCUUCUCCAAC
GUCACCUGGUUUCAUGCUAUUCAUGUGUCAGGGACCAACGGAACUAAAAGGUUCGA
CAAUCCCGUACUGCCUUUCAACGACGGGGUGUAUUUCGCAUCAACAGAAAAAAGCAA
CAUUAUCAGAGGCUGGAUUUUUGGCACUACACUCGACUCAAAGACCCAAAGUUUGCU
UAUUGUUAAUAACGCAACGAAUGUCGUAAUUAAAGUCUGUGAAUUCCAGUUUUGUAA
CGACCCAUUUCUGGAUGUAUACUAUCAUAAGAACAAUAAGUCCUGGAUGGAGUCAGA
GUUCAGAGUCUACUCAUCAGCAAACAAUUGCACUUUUGAGUACGUAUCUCAGCCAUU
UCUGAUGGACCUGGAAGGCAAGCAAGGGAACUUCAAAAAUCUCCGAGAGUUUGUUU
UCAAAAACAUAGAUGGUUACUUCAAGAUCUAUUCUAAACACACCCCGAUUAAUCUUG
GCCGGGACCUGCCCCAGGGUUUCUCUGCCCUCGAGCCCCUUGUAGACUUGCCCAUU
GGGAUAAAUAUUACUAGGUUUCAGACACUGCUUGCGCUGCACAGGUCAUACCUCACU
CCUGGGGAUUCAUCAAGCGGCUGGACUGCAGGUGCCGCUGCAUACUACGUGGGAUA
CCUCCAACCCCGAACUUUUUUGCUCAAGUACAAUGAGAAUGGGACGAUAACUGAUGC
CGUAGACUGCGCACUCGAUCCUCUGUCUGAGACGAAGUGCACACUGAAGAGUUUCA
CGGUGGAAAAGGGUAUAUAUCAAACAUCCAAUUUCCGAGUCCAGCCCACUGAAAGUA
UUGUUAGGUUCCCGAACAUCACAAAUCUCUGUCCAUUUGACGAGGUAUUUAACGCGA
CACGCUUUGCUUCCGUGUAUGCGUGGAAUAGAAAGCGCAUUAGCAACUGUGUCGCG
GAUUAUUCAGUCUUGUACAACUUUGCGCCUUUUUUCGCAUUUAAAUGCUACGGGGU
CAGCCCCACAAAGCUGAAUGAUCUCUGCUUCACAAAUGUAUAUGCAGAUAGCUUUGU
CAUCCGAGGUAACGAAGUUUCUCAGAUUGCUCCCGGCCAAACGGGAAACAUAGCUGA
CUAUAACUAUAAGCUCCCAGACGAUUUUACGGGUUGUGUCAUUGCAUGGAACUCCAA
UAAAUUGGAUUCAAAGGUAGGUGGUAAUUAUAACUACAGGUAUAGGCUGUUCAGGAA
GUCAAACCUGAAACCGUUUGAACGAGAUAUCUCAACUGAGAUUUAUCAAGCGGGCAA
UAAACCCUGCAACGGAGUCGCGGGUGUUAAUUGCUACUUCCCAUUGCAGAGUUACG
GCUUUCGGCCGACUUAUGGCGUCGGACAUCAACCUUACAGGGUAGUUGUUCUGUCU
UUCGAACUCCUGCAUGCUCCGGCAACGGUGUGUGGUCCCAAGAAAAGUACUAACCU
UGUCAAAAAUAAGUGUGUGAACUUCAAUUUUAACGGUCUUACAGGUACUGGAGUAUU
GACUGAGUCAAAUAAGAAGUUUCUUCCAUUCCAACAAUUCGGCCGCGAUAUAGCUGA
UACAACGGAUGCCGUCCGAGACCCUCAGACUCUGGAGAUCCUGGACAUAACUCCAU
GUUCUUUUGGAGGCGUUAGUGUGAUUACCCCAGGCACUAAUACCUCAAACCAAGUA
GCCGUCCUGUAUCAAGGAGUCAAUUGUACUGAGGUCCCUGUGGCCAUCCAUGCGGA
CCAACUGACACCCACAUGGCGCGUAUAUAGCACAGGAUCAAACGUAUUCCAGACUCG
CGCGGGUUGUCUGAUCGGGGCGGAAUACGUAAACAAUAGCUAUGAGUGUGAUAUAC
CUAUUGGAGCUGGGAUUUGCGCAAGCUAUCAAACACAAACGAAAUCCCACGGAUCGG
CAUCAUCAGUUGCAUCUCAAUCUAUUAUCGCUUAUACAAUGUCUCUUGGCGCUGAGA
ACUCCGUUGCUUACAGCAAUAAUAGCAUUGCCAUACCGACGAACUUUACUAUUAGCG
UAACGACUGAGAUACUCCCGGUGUCUAUGACAAAAACUAGCGUUGAUUGUACAAUGU
ACAUUUGUGGGGAUAGUACAGAGUGCUCAAACUUGCUUCUCCAGUACGGGUCAUUC
UGUACCCAACUUAAACGCGCAUUGACUGGUAUUGCCGUCGAGCAAGACAAAAACACA
CAAGAGGUUUUCGCUCAAGUAAAACAAAUUUACAAAACGCCCCCUAUAAAAUAUUUUG
GCGGUUUCAACUUUUCUCAAAUUCUGCCCGAUCCUUCUAAGCCUAGUAAGCGAAGCU
UCAUUGAAGACCUGCUUUUCAACAAAGUAACCUUGGCUGACGCGGGUUUCAUAAAAC
AGUAUGGAGACUGUCUCGGGGACAUAGCCGCAAGGGACUUGAUUUGUGCCCAAAAA
UUCAACGGACUGACAGUUCUUCCUCCACUUUUGACUGAUGAGAUGAUAGCACAAUAC
ACUUCUGCUUUGCUGGCCGGUACAAUAACGAGUGGGUGGACCUUCGGGGCUGGGG
CUGCCCUCCAAAUACCCUUCGCUAUGCAAAUGGCAUAUCGAUUUAAUGGCAUCGGG
GUUACACAGAACGUCCUGUAUGAAAACCAAAAGCUCAUUGCCAACCAGUUUAACUCA
GCAAUCGGAAAAAUUCAGGACAGCCUGAGUAGUACCGCCAGCGCCUUGGGAAAGUU
GCAGGAUGUCGUCAACCAUAACGCCCAAGCAUUGAAUACGUUGGUCAAACAGCUCUC
CUCUAAGUUCGGUGCUAUAUCCUCAGUCCUCAACGAUAUCCUGAGCAGGCUCGAUC
CCCCUGAGGCCGAAGUGCAGAUCGAUCGGCUGAUCACUGGUAGGCUGCAGAGUCUG
CAGACAUAUGUCACCCAGCAGCUGAUCAGAGCAGCCGAGAUUCGAGCUUCAGCUAAC
UUAGCUGCCACAAAGAUGAGUGAGUGCGUGCUGGGCCAGUCCAAGAGGGUGGAUUU
CUGCGGAAAAGGCUAUCAUCUCAUGUCCUUCCCCCAAAGCGCACCCCACGGGGUUG
UCUUCCUGCACGUAACGUAUGUGCCCGCUCAGGAGAAGAACUUUACUACUGCCCCC
GCCAUCUGCCACGAUGGGAAAGCUCACUUCCCGAGAGAAGGAGUAUUCGUUUCCAA
UGGGACGCAUUGGUUCGUCACCCAGCGGAACUUUUACGAACCACAGAUCAUUACCAC
GGAUAAUACAUUUGUGAGCGGGAAUUGCGAUGUGGUGAUUGGAAUUGUAAAUAACA
CAGUGUACGAUCCACUGCAGUCAGAAUUAGACUCAAUUAAGGAGGAGUUGGAUAAGA
UUCAUAAAAAUGGUUCAGGUGGAUCAGGUGAUAUAGAAAAACUCCUCAAUGAACAAG
UAAAUAAGGAGAUGCAAAGUUCUAACCUGUACAUGAGCAUGUCUUCUUGGUGUUACA
CCCAUAGCCUCGAUGGAGCGGGAUUGUUCCUUUUUGACCACGCUGCGGAGGAGUAU
GAGCAUGCUAAAAAGCUGAUAAUAUUUCUCAACGAGAAUAAUGUUCCAGUGCAAUUG
ACAAGUAUAUCCGCCCCUGAGCAUAAGUUUGAAGGGCUCACACAAAUUUUCCAAAAG
GCAUACGAACACGAACAGCACAUUAGCGAGUCUAUUAACAACAUUGUUGAUCAUGCA
AUCAAGUCCAAAGAUCACGCCACGUUUAAUUUCCUCCAGUGGUAUGUAGCUGAGCAA
CAUGAGGAAGAAGUGUUGUUUAAGGAUAUUCUUGAUAAAAUUGAACUUAUUGGAAAU
GAGAACCAUGGCCUCUAUCUUGCGGACCAAUACGUCAAGGGAAUUGCCAAGUCCCG
CAAGAGUUGAUGAUAAUAGGACUAGUGGAUCCAACUGGAGACUGGGUGAAAGUGAC
UACCAGAAAGUGAGGAAGCCUAAAUAAACCUAGCGUACGUAAAAAAUGGAAAGAACC
UAGCGUACGAAAAAAAAAAAAAAAAAAAAAAAAAAAAAAAAAAAAAAAAAAAAAAAAAAA
AAAAAAAAAAAAAAAAAAAAAAAAAA
SARS-CoV2 Wuhan D614G Spike-ferritin fusion protein polypeptide
sequence (SEQ ID NO: 12)
MPLLLLLPLLWAGALASQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQSLLIVN
NATNVVIKVCEFQFCNDPFLGVYYHKNNKSWMESEFRVYSSANNCTFEYVSQPFLMDLEG
KQGNFKNLREFVFKNIDGYFKIYSKHTPINLVRDLPQGFSALEPLVDLPIGINITRFQTLLALH
RSYLTPGDSSSGWTAGAAAYYVGYLQPRTFLLKYNENGTITDAVDCALDPLSETKCTLKSF
TVEKGIYQTSNFRVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTPCNGVEGENCYFP
LQSYGFQPTNGVGYQPYRVVVLSFELLHAPATVCGPKKSTNLVKNKCVNFNFNGLTGTGV
LTESNKKFLPFQQFGRDIADTTDAVRDPQTLEILDITPCSFGGVSVITPGTNTSNQVAVLYQG
VNCTEVPVAIHADQLTPTWRVYSTGSNVFQTRAGCLIGAEHVNNSYECDIPIGAGICASYQT
QTNSPGSASSVASQSIIAYTMSLGAENSVAYSNNSIAIPTNFTISVTTEILPVSMTKTSVDCTM
YICGDSTECSNLLLQYGSFCTQLNRALTGIAVEQDKNTQEVFAQVKQIYKTPPIKDFGGFNF
SQILPDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQFN
SAIGKIQDSLSSTASALGKLQDVVNQNAQALNTLVKQLSSNFGAISSVLNDILSRLDPPEAEV
QIDRLITGRLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTQRNFYEPQI
ITTDNTFVSGNCDVVIGIVNNTVYDPLQSELDSIKEELDKIHKN
SARS-CoV-2 Delta Spike-ferritin fusion protein polypeptide sequence
(SEQ ID NO: 13)
MPLLLLLPLLWAGALASQCVNLITRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLMDLEG
KQGNFKNLREFVFKNIDGYFKIYSKHTPINLGRDLPQGFSALEPLVDLPIGINITRFQTLLALH
RSYLTPGDSSSGWTAGAAAYYVGYLQPRTFLLKYNENGTITDAVDCALDPLSETKCTLKSF
TVEKGIYQTSNFRVQPTESIVRFPNITNLCPFDEVFNATRFASVYAWNRKRISNCVADYSVL
YNFAPFFAFKCYGVSPTKLNDLCFTNVYADSFVIRGNEVSQIAPGQTGNIADYNYKLPDDFT
GCVIAWNSNKLDSKVGGNYNYRYRLFRKSNLKPFERDISTEIYQAGNKPCNGVAGVNCYFP
LQSYGFRPTYGVGHQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGTNTSNQVAVLYQG
VNCTEVPVAIHADQLTPTWRVYSTGSNVFQTRAGCLIGAEYVNNSYECDIPIGAGICASYQT
QTKSHGSASSVASQSIIAYTMSLGAENSVAYSNNSIAIPTNFTISVTTEILPVSMTKTSVDCTM
YICGDSTECSNLLLQYGSFCTQLKRALTGIAVEQDKNTQEVFAQVKQIYKTPPIKYFGGFNF
SQILPDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQFN
SAIGKIQDSLSSTASALGKLQDVVNHNAQALNTLVKQLSSKFGAISSVLNDILSRLDPPEAEV
QIDRLITGRLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTQRNFYEPQI
ITTDNTFVSGNCDVVIGIVNNTVYDPLQSELDSIKEELDKIHKN
SARS-CoV-2 Omicron BA.4/5 Spike-ferritin fusion protein polypeptide
sequence (SEQ ID NO: 14) (leader sequence underlined)
MPLLLLLPLLWAGALASQCVNLITRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLMDLEG
KQGNFKNLREFVFKNIDGYFKIYSKHTPINLGRDLPQGFSALEPLVDLPIGINITRFQTLLALH
RSYLTPGDSSSGWTAGAAAYYVGYLQPRTFLLKYNENGTITDAVDCALDPLSETKCTLKSF
TVEKGIYQTSNFRVQPTESIVRFPNITNLCPFDEVFNATRFASVYAWNRKRISNCVADYSVL
YNFAPFFAFKCYGVSPTKLNDLCFTNVYADSFVIRGNEVSQIAPGQTGNIADYNYKLPDDFT
GCVIAWNSNKLDSKVGGNYNYRYRLFRKSNLKPFERDISTEIYQAGNKPCNGVAGVNCYFP
LQSYGFRPTYGVGHQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGTNTSNQVAVLYQG
VNCTEVPVAIHADQLTPTWRVYSTGSNVFQTRAGCLIGAEYVNNSYECDIPIGAGICASYQT
QTKSHGSASSVASQSIIAYTMSLGAENSVAYSNNSIAIPTNFTISVTTEILPVSMTKTSVDCTM
YICGDSTECSNLLLQYGSFCTQLKRALTGIAVEQDKNTQEVFAQVKQIYKTPPIKYFGGFNF
SQILPDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQFN
SAIGKIQDSLSSTASALGKLQDVVNHNAQALNTLVKQLSSKFGAISSVLNDILSRLDPPEAEV
QIDRLITGRLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTQRNFYEPQI
ITTDNTFVSGNCDVVIGIVNNTVYDPLQSELDSIKEELDKIHKNGSGGSGDIEKLLNEQVNKE
MQSSNLYMSMSSWCYTHSLDGAGLFLFDHAAEEYEHAKKLIIFLNENNVPVQLTSISAPEHK
FEGLTQIFQKAYEHEQHISESINNIVDHAIKSKDHATFNFLQWYVAEQHEEEVLFKDILDKIELI
GNENHGLYLADQYVKGIAKSRKS
T7 Promoter (SEQ ID NO: 15) TAATACGACTCACTATAAGG
SARS-CoV-2 XBB.1.5 Spike Protein-linker-ferritin RNA sequence (SEQ
ID NO: 16)
AUGCCCCUCCUUCUCCUUCUUCCCCUCCUUUGGGCUGGAGCGCUGGCCUCUCAGUG
UGUAAAUCUCAUUACCCGCACGCAGAGCUAUACCAAUUCCUUCACACGGGGCGUGUA
CUAUCCCGACAAGGUGUUUAGAUCUAGCGUGCUGCACUCCACACAGGAUCUGUUUC
UGCCUUUCUUUUCUAACGUGACCUGGUUCCACGCCAUCCACGUGAGCGGCACCAAU
GGCACAAAGAGGUUCGACAAUCCAGCCCUGCCCUUUAACGAUGGCGUGUACUUCGC
CUCCACCGAGAAGUCUAACAUCAUCCGCGGCUGGAUCUUUGGCACCACACUGGACA
GCAAGACACAGUCCCUGCUGAUCGUGAACAAUGCCACCAACGUGGUCAUCAAGGUG
UGCGAGUUCCAGUUUUGUAAUGAUCCAUUCCUGGACGUGUAUCAGAAGAACAAUAAG
UCUUGGAUGGAGAGCGAGUUCCGGGUGUAUUCCUCUGCCAACAAUUGCACAUUUGA
GUACGUGUCCCAGCCCUUCCUGAUGGACCUGGAGGGCAAGGAGGGCAAUUUCAAGA
ACCUGCGGGAGUUCGUGUUUAAGAAUAUCGAUGGCUACUUCAAGAUCUACUCCAAG
CACACCCCCAUCAACCUGGAGCGGGACCUGCCACAGGGCUUCUCUGCCCUGGAGCC
ACUGGUGGAUCUGCCCAUCGGCAUCAACAUCACCCGGUUUCAGACACUGCUGGCCC
UGCACAGAAGCUACCUGACACCAGUGGACAGCUCCUCUGGCUGGACCGCAGGAGCA
GCAGCCUACUAUGUGGGCUAUCUGCAGCCCCGGACCUUCCUGCUGAAGUACAACGA
GAAUGGCACCAUCACAGACGCCGUGGAUUGCGCCCUGGAUCCCCUGUCUGAGACAA
AGUGUACACUGAAGAGUUUUACCGUGGAGAAGGGCAUCUAUCAGACAAGCAAUUUCA
GGGUGCAGCCUACCGAGUCCAUCGUGCGCUUUCCCAAUAUCACAAACCUGUGCCCU
UUUCACGAGGUGUUCAACGCCACCACCUUCGCCAGCGUGUACGCCUGGAAUAGGAA
GCGCAUCUCCAACUGCGUGGCCGACUAUUCUGUGAUCUACAACUUCGCCCCCUUCU
UCGCCUUUAAGUGCUAUGGCGUGAGCCCCACAAAGCUGAAUGACCUGUGCUUUACC
AACGUGUACGCCGAUUCCUUCGUGAUCAGGGGCAACGAGGUGAGCCAGAUCGCACC
AGGACAGACAGGCAACAUCGCAGACUACAAUUAUAAGCUGCCUGACGAUUUCACCGG
CUGCGUGAUCGCCUGGAACUCUAACAAGCUGGAUAGCAAACCCAGCGGCAACUACAA
UUAUCUGUACCGGCUGUUUAGAAAGUCUAAGCUGAAGCCAUUCGAGCGGGACAUCU
CCACAGAGAUCUACCAGGCCGGCAACAAGCCCUGCAAUGGCGUGGCCGGCCCAAAC
UGUUAUAGCCCAUUGCAGAGUUACGGCUUUCGGCCGACUUAUGGCGUCGGACAUCA
ACCUUACAGGGUAGUUGUUCUGUCUUUCGAACUCCUGCAUGCUCCGGCAACGGUGU
GUGGUCCCAAGAAAAGUACUAACCUUGUCAAAAAUAAGUGUGUGAACUUCAAUUUUA
ACGGUCUUACAGGUACUGGAGUAUUGACUGAGUCAAAUAAGAAGUUUCUUCCAUUCC
AACAAUUCGGCCGCGAUAUAGCUGAUACAACGGAUGCCGUCCGAGACCCUCAGACUC
UGGAGAUCCUGGACAUAACUCCAUGUUCUUUUGGAGGCGUUAGUGUGAUUACCCCA
GGCACUAAUACCUCAAACCAAGUAGCCGUCCUGUAUCAAGGAGUCAAUUGUACUGAG
GUCCCUGUGGCCAUCCAUGCGGACCAACUGACACCCACAUGGCGCGUAUAUAGCAC
AGGAUCAAACGUAUUCCAGACUCGCGCGGGUUGUCUGAUCGGGGCGGAAUACGUAA
ACAAUAGCUAUGAGUGUGAUAUACCUAUUGGAGCUGGGAUUUGCGCAAGCUAUCAAA
CACAAACGAAAUCCCACGGAUCGGCAUCAUCAGUUGCAUCUCAAUCUAUUAUCGCUU
AUACAAUGUCUCUUGGCGCUGAGAACUCCGUUGCUUACAGCAAUAAUAGCAUUGCCA
UACCGACGAACUUUACUAUUAGCGUAACGACUGAGAUACUCCCGGUGUCUAUGACAA
AAACUAGCGUUGAUUGUACAAUGUACAUUUGUGGGGAUAGUACAGAGUGCUCAAACU
UGCUUCUCCAGUACGGGUCAUUCUGUACCCAACUUAAACGCGCAUUGACUGGUAUU
GCCGUCGAGCAAGACAAAAACACACAAGAGGUUUUCGCUCAAGUAAAACAAAUUUAC
AAAACGCCCCCUAUAAAAUAUUUUGGCGGUUUCAACUUUUCUCAAAUUCUGCCCGAU
CCUUCUAAGCCUAGUAAGCGAAGCUUCAUUGAAGACCUGCUUUUCAACAAAGUAACC
UUGGCUGACGCGGGUUUCAUAAAACAGUAUGGAGACUGUCUCGGGGACAUAGCCGC
AAGGGACUUGAUUUGUGCCCAAAAAUUCAACGGACUGACAGUUCUUCCUCCACUUUU
GACUGAUGAGAUGAUAGCACAAUACACUUCUGCUUUGCUGGCCGGUACAAUAACGAG
UGGGUGGACCUUCGGGGCUGGGGCUGCCCUCCAAAUACCCUUCGCUAUGCAAAUGG
CAUAUCGAUUUAAUGGCAUCGGGGUUACACAGAACGUCCUGUAUGAAAACCAAAAGC
UCAUUGCCAACCAGUUUAACUCAGCAAUCGGAAAAAUUCAGGACAGCCUGAGUAGUA
CCGCCAGCGCCUUGGGAAAGUUGCAGGAUGUCGUCAACCAUAACGCCCAAGCAUUG
AAUACGUUGGUCAAACAGCUCUCCUCUAAGUUCGGUGCUAUAUCCUCAGUCCUCAAC
GAUAUCCUGAGCAGGCUCGAUCCCCCUGAGGCCGAAGUGCAGAUCGAUCGGCUGAU
CACUGGUAGGCUGCAGAGUCUGCAGACAUAUGUCACCCAGCAGCUGAUCAGAGCAG
CCGAGAUUCGAGCUUCAGCUAACUUAGCUGCCACAAAGAUGAGUGAGUGCGUGCUG
GGCCAGUCCAAGAGGGUGGAUUUCUGCGGAAAAGGCUAUCAUCUCAUGUCCUUCCC
CCAAAGCGCACCCCACGGGGUUGUCUUCCUGCACGUAACGUAUGUGCCCGCUCAGG
AGAAGAACUUUACUACUGCCCCCGCCAUCUGCCACGAUGGGAAAGCUCACUUCCCGA
GAGAAGGAGUAUUCGUUUCCAAUGGGACGCAUUGGUUCGUCACCCAGCGGAACUUU
UACGAACCACAGAUCAUUACCACGGAUAAUACAUUUGUGAGCGGGAAUUGCGAUGUG
GUGAUUGGAAUUGUAAAUAACACAGUGUACGAUCCACUGCAGUCAGAAUUAGACUCA
AUUAAGGAGGAGUUGGAUAAGAUUCAUAAAAAUGGUUCAGGUGGAUCAGGUGAUAUA
GAAAAACUCCUCAAUGAACAAGUAAAUAAGGAGAUGCAAAGUUCUAACCUGUACAUGA
GCAUGUCUUCUUGGUGUUACACCCAUAGCCUCGAUGGAGCGGGAUUGUUCCUUUUU
GACCACGCUGCGGAGGAGUAUGAGCAUGCUAAAAAGCUGAUAAUAUUUCUCAACGAG
AAUAAUGUUCCAGUGCAAUUGACAAGUAUAUCCGCCCCUGAGCAUAAGUUUGAAGGG
CUCACACAAAUUUUCCAAAAGGCAUACGAACACGAACAGCACAUUAGCGAGUCUAUU
AACAACAUUGUUGAUCAUGCAAUCAAGUCCAAAGAUCACGCCACGUUUAAUUUCCUC
CAGUGGUAUGUAGCUGAGCAACAUGAGGAAGAAGUGUUGUUUAAGGAUAUUCUUGA
UAAAAUUGAACUUAUUGGAAAUGAGAACCAUGGCCUCUAUCUUGCGGACCAAUACGU
CAAGGGAAUUGCCAAGUCCCGCAAGAGU
mRNA construct sequence encoding SARS-CoV-2 XBB.1.5 Spike-ferritin
fusion protein (SEQ ID NO: 17)
AGGAUUGUGCUGCAUCAAGCUUGCCGCCACCAUGCCCCUCCUUCUCCUUCUUCCCC
UCCUUUGGGCUGGAGCGCUGGCCUCUCAGUGUGUAAAUCUCAUUACCCGCACGCAG
AGCUAUACCAAUUCCUUCACACGGGGCGUGUACUAUCCCGACAAGGUGUUUAGAUC
UAGCGUGCUGCACUCCACACAGGAUCUGUUUCUGCCUUUCUUUUCUAACGUGACCU
GGUUCCACGCCAUCCACGUGAGCGGCACCAAUGGCACAAAGAGGUUCGACAAUCCA
GCCCUGCCCUUUAACGAUGGCGUGUACUUCGCCUCCACCGAGAAGUCUAACAUCAU
CCGCGGCUGGAUCUUUGGCACCACACUGGACAGCAAGACACAGUCCCUGCUGAUCG
UGAACAAUGCCACCAACGUGGUCAUCAAGGUGUGCGAGUUCCAGUUUUGUAAUGAU
CCAUUCCUGGACGUGUAUCAGAAGAACAAUAAGUCUUGGAUGGAGAGCGAGUUCCG
GGUGUAUUCCUCUGCCAACAAUUGCACAUUUGAGUACGUGUCCCAGCCCUUCCUGA
UGGACCUGGAGGGCAAGGAGGGCAAUUUCAAGAACCUGCGGGAGUUCGUGUUUAAG
AAUAUCGAUGGCUACUUCAAGAUCUACUCCAAGCACACCCCCAUCAACCUGGAGCGG
GACCUGCCACAGGGCUUCUCUGCCCUGGAGCCACUGGUGGAUCUGCCCAUCGGCAU
CAACAUCACCCGGUUUCAGACACUGCUGGCCCUGCACAGAAGCUACCUGACACCAGU
GGACAGCUCCUCUGGCUGGACCGCAGGAGCAGCAGCCUACUAUGUGGGCUAUCUGC
AGCCCCGGACCUUCCUGCUGAAGUACAACGAGAAUGGCACCAUCACAGACGCCGUG
GAUUGCGCCCUGGAUCCCCUGUCUGAGACAAAGUGUACACUGAAGAGUUUUACCGU
GGAGAAGGGCAUCUAUCAGACAAGCAAUUUCAGGGUGCAGCCUACCGAGUCCAUCG
UGCGCUUUCCCAAUAUCACAAACCUGUGCCCUUUUCACGAGGUGUUCAACGCCACCA
CCUUCGCCAGCGUGUACGCCUGGAAUAGGAAGCGCAUCUCCAACUGCGUGGCCGAC
UAUUCUGUGAUCUACAACUUCGCCCCCUUCUUCGCCUUUAAGUGCUAUGGCGUGAG
CCCCACAAAGCUGAAUGACCUGUGCUUUACCAACGUGUACGCCGAUUCCUUCGUGA
UCAGGGGCAACGAGGUGAGCCAGAUCGCACCAGGACAGACAGGCAACAUCGCAGAC
UACAAUUAUAAGCUGCCUGACGAUUUCACCGGCUGCGUGAUCGCCUGGAACUCUAA
CAAGCUGGAUAGCAAACCCAGCGGCAACUACAAUUAUCUGUACCGGCUGUUUAGAAA
GUCUAAGCUGAAGCCAUUCGAGCGGGACAUCUCCACAGAGAUCUACCAGGCCGGCA
ACAAGCCCUGCAAUGGCGUGGCCGGCCCAAACUGUUAUAGCCCAUUGCAGAGUUAC
GGCUUUCGGCCGACUUAUGGCGUCGGACAUCAACCUUACAGGGUAGUUGUUCUGUC
UUUCGAACUCCUGCAUGCUCCGGCAACGGUGUGUGGUCCCAAGAAAAGUACUAACC
UUGUCAAAAAUAAGUGUGUGAACUUCAAUUUUAACGGUCUUACAGGUACUGGAGUAU
UGACUGAGUCAAAUAAGAAGUUUCUUCCAUUCCAACAAUUCGGCCGCGAUAUAGCUG
AUACAACGGAUGCCGUCCGAGACCCUCAGACUCUGGAGAUCCUGGACAUAACUCCAU
GUUCUUUUGGAGGCGUUAGUGUGAUUACCCCAGGCACUAAUACCUCAAACCAAGUA
GCCGUCCUGUAUCAAGGAGUCAAUUGUACUGAGGUCCCUGUGGCCAUCCAUGCGGA
CCAACUGACACCCACAUGGCGCGUAUAUAGCACAGGAUCAAACGUAUUCCAGACUCG
CGCGGGUUGUCUGAUCGGGGCGGAAUACGUAAACAAUAGCUAUGAGUGUGAUAUAC
CUAUUGGAGCUGGGAUUUGCGCAAGCUAUCAAACACAAACGAAAUCCCACGGAUCGG
CAUCAUCAGUUGCAUCUCAAUCUAUUAUCGCUUAUACAAUGUCUCUUGGCGCUGAGA
ACUCCGUUGCUUACAGCAAUAAUAGCAUUGCCAUACCGACGAACUUUACUAUUAGCG
UAACGACUGAGAUACUCCCGGUGUCUAUGACAAAAACUAGCGUUGAUUGUACAAUGU
ACAUUUGUGGGGAUAGUACAGAGUGCUCAAACUUGCUUCUCCAGUACGGGUCAUUC
UGUACCCAACUUAAACGCGCAUUGACUGGUAUUGCCGUCGAGCAAGACAAAAACACA
CAAGAGGUUUUCGCUCAAGUAAAACAAAUUUACAAAACGCCCCCUAUAAAAUAUUUUG
GCGGUUUCAACUUUUCUCAAAUUCUGCCCGAUCCUUCUAAGCCUAGUAAGCGAAGCU
UCAUUGAAGACCUGCUUUUCAACAAAGUAACCUUGGCUGACGCGGGUUUCAUAAAAC
AGUAUGGAGACUGUCUCGGGGACAUAGCCGCAAGGGACUUGAUUUGUGCCCAAAAA
UUCAACGGACUGACAGUUCUUCCUCCACUUUUGACUGAUGAGAUGAUAGCACAAUAC
ACUUCUGCUUUGCUGGCCGGUACAAUAACGAGUGGGUGGACCUUCGGGGCUGGGG
CUGCCCUCCAAAUACCCUUCGCUAUGCAAAUGGCAUAUCGAUUUAAUGGCAUCGGG
GUUACACAGAACGUCCUGUAUGAAAACCAAAAGCUCAUUGCCAACCAGUUUAACUCA
GCAAUCGGAAAAAUUCAGGACAGCCUGAGUAGUACCGCCAGCGCCUUGGGAAAGUU
GCAGGAUGUCGUCAACCAUAACGCCCAAGCAUUGAAUACGUUGGUCAAACAGCUCUC
CUCUAAGUUCGGUGCUAUAUCCUCAGUCCUCAACGAUAUCCUGAGCAGGCUCGAUC
CCCCUGAGGCCGAAGUGCAGAUCGAUCGGCUGAUCACUGGUAGGCUGCAGAGUCUG
CAGACAUAUGUCACCCAGCAGCUGAUCAGAGCAGCCGAGAUUCGAGCUUCAGCUAAC
UUAGCUGCCACAAAGAUGAGUGAGUGCGUGCUGGGCCAGUCCAAGAGGGUGGAUUU
CUGCGGAAAAGGCUAUCAUCUCAUGUCCUUCCCCCAAAGCGCACCCCACGGGGUUG
UCUUCCUGCACGUAACGUAUGUGCCCGCUCAGGAGAAGAACUUUACUACUGCCCCC
GCCAUCUGCCACGAUGGGAAAGCUCACUUCCCGAGAGAAGGAGUAUUCGUUUCCAA
UGGGACGCAUUGGUUCGUCACCCAGCGGAACUUUUACGAACCACAGAUCAUUACCAC
GGAUAAUACAUUUGUGAGCGGGAAUUGCGAUGUGGUGAUUGGAAUUGUAAAUAACA
CAGUGUACGAUCCACUGCAGUCAGAAUUAGACUCAAUUAAGGAGGAGUUGGAUAAGA
UUCAUAAAAAUGGUUCAGGUGGAUCAGGUGAUAUAGAAAAACUCCUCAAUGAACAAG
UAAAUAAGGAGAUGCAAAGUUCUAACCUGUACAUGAGCAUGUCUUCUUGGUGUUACA
CCCAUAGCCUCGAUGGAGCGGGAUUGUUCCUUUUUGACCACGCUGCGGAGGAGUAU
GAGCAUGCUAAAAAGCUGAUAAUAUUUCUCAACGAGAAUAAUGUUCCAGUGCAAUUG
ACAAGUAUAUCCGCCCCUGAGCAUAAGUUUGAAGGGCUCACACAAAUUUUCCAAAAG
GCAUACGAACACGAACAGCACAUUAGCGAGUCUAUUAACAACAUUGUUGAUCAUGCA
AUCAAGUCCAAAGAUCACGCCACGUUUAAUUUCCUCCAGUGGUAUGUAGCUGAGCAA
CAUGAGGAAGAAGUGUUGUUUAAGGAUAUUCUUGAUAAAAUUGAACUUAUUGGAAAU
GAGAACCAUGGCCUCUAUCUUGCGGACCAAUACGUCAAGGGAAUUGCCAAGUCCCG
CAAGAGUUGAUGAUAAUAGGACUAGUGGAUCCAACUGGAGACUGGGUGAAAGUGAC
UACCAGAAAGUGAGGAAGCCUAAAUAAACCUAGCGUACGUAAAAAAUGGAAAGAACC
UAGCGUACGAAAAAAAAAAAAAAAAAAAAAAAAAAAAAAAAAAAAAAAAAAAAAAAAAAA
AAAAAAAAAAAAAAAAAAAAAAA
SARS-CoV2 Omicron XBB.1.5 Spike-ferritin fusion protein polypeptide
sequence (leaders equence underlined) (SEQ ID NO: 18)
MPLLLLLPLLWAGALASQCVNLITRTQSYTNSFTRGVYYPDKVFRSSVLHSTQDLFLPFFSN
VTWFHAIHVSGTNGTKRFDNPALPFNDGVYFASTEKSNIIRGWIFGTTLDSKTQSLLIVNNAT
NVVIKVCEFQFCNDPFLDVYQKNNKSWMESEFRVYSSANNCTFEYVSQPFLMDLEGKEGN
FKNLREFVFKNIDGYFKIYSKHTPINLERDLPQGFSALEPLVDLPIGINITRFQTLLALHRSYLT
PVDSSSGWTAGAAAYYVGYLQPRTFLLKYNENGTITDAVDCALDPLSETKCTLKSFTVEKGI
YQTSNFRVQPTESIVRFPNITNLCPFHEVFNATTFASVYAWNRKRISNCVADYSVIYNFAPFF
AFKCYGVSPTKLNDLCFTNVYADSFVIRGNEVSQIAPGQTGNIADYNYKLPDDFTGCVIAWN
SNKLDSKPSGNYNYLYRLFRKSKLKPFERDISTEIYQAGNKPCNGVAGPNCYSPLQSYGFR
PTYGVGHQPYRVVVLSFELLHAPATVCGPKKSTNLVKNKCVNFNFNGLTGTGVLTESNKKF
LPFQQFGRDIADTTDAVRDPQTLEILDITPCSFGGVSVITPGTNTSNQVAVLYQGVNCTEVP
VAIHADQLTPTWRVYSTGSNVFQTRAGCLIGAEYVNNSYECDIPIGAGICASYQTQTKSHGS
ASSVASQSIIAYTMSLGAENSVAYSNNSIAIPTNFTISVTTEILPVSMTKTSVDCTMYICGDST
ECSNLLLQYGSFCTQLKRALTGIAVEQDKNTQEVFAQVKQIYKTPPIKYFGGFNFSQILPDPS
KPSKRSFIEDLLFNKVTLADAGFIKQYGDCLGDIAARDLICAQKFNGLTVLPPLLTDEMIAQYT
SALLAGTITSGWTFGAGAALQIPFAMQMAYRFNGIGVTQNVLYENQKLIANQFNSAIGKIQD
SLSSTASALGKLQDVVNHNAQALNTLVKQLSSKFGAISSVLNDILSRLDPPEAEVQIDRLITG
RLQSLQTYVTQQLIRAAEIRASANLAATKMSECVLGQSKRVDFCGKGYHLMSFPQSAPHGV
VFLHVTYVPAQEKNFTTAPAICHDGKAHFPREGVFVSNGTHWFVTQRNFYEPQIITTDNTFV
SGNCDVVIGIVNNTVYDPLQSELDSIKEELDKIHKNGSGGSGDIEKLLNEQVNKEMQSSNLY
MSMSSWCYTHSLDGAGLFLFDHAAEEYEHAKKLIIFLNENNVPVQLTSISAPEHKFEGLTQIF
QKAYEHEQHISESINNIVDHAIKSKDHATFNFLQWYVAEQHEEEVLFKDILDKIELIGNENHGL
YLADQYVKGIAKSRKS

1. A method of inducing a pan-sarbecoronavirus variant immune response in an individual who has previously received one or more doses of a first SARS-COV-2 vaccine directed against a first sarbecoronavirus variant or variants (Variant 1), said method comprising administering to said individual one or more doses of a second SARS-COV-2 vaccine,
wherein the second SARS-COV-2 vaccine comprises a mRNA encoding a single Spike (S) protein, or an immunogenic fragment or immunogenic variant thereof, derived from a sarbecoronavirus variant (Variant 2) that is different to Variant 1,
wherein the S protein is encoded as a S protein-multimerization subunit fusion, and
wherein the method induces a pan-variant immune response in the individual against sarbecoronavirus Variant 1 and Variant 2 and induces an immune response against one or more additional sarbecoronavirus variants that are different from Variant 1 and Variant 2.

2. A method according to claim 1, wherein the S protein-multimerization subunit fusion is a protein-ferritin subunit fusion.

3. A method according to claim 1, wherein the mRNA of the second SARS-COV-2 vaccine is formulated in a lipid nanoparticle (LNP).

4. A method according to claim 1, wherein Variant 2 is SARS-COV-2 Omicron.

5. (canceled)

6. (canceled)

7. (canceled)

8. A method according to claim 1, wherein the S protein or immunogenic fragment or immunogenic variant thereof comprises K986P and/or V987P mutations.

9. (canceled)

10. (canceled)

11. A method according to claim 1, wherein the first SARS-COV-2 vaccine does not comprise a nucleic acid encoding a sarbecoronavirus S protein in the form of a fusion protein that is capable of assembling to form a nanoparticle in vivo.

12. A method according to claim 1, wherein the method induces an immune response against one or more additional sarbecoronavirus variants that are mutationally diverse from Variant 1 and/or Variant 2.

13. A method according to claim 1, wherein the second SARS-COV-2 vaccine is a monovalent vaccine.

14. A method according to claim 1 wherein the second SARS-COV-2 vaccine is a bivalent vaccine comprising a further mRNA encoding a single Spike (S) protein, or an immunogenic fragment or immunogenic variant thereof, wherein the S protein is encoded as a S protein-multimerization subunit fusion and is derived from a sarbecoronavirus variant that is different to Variant 2 and is the same as or different to Variant 1.

15. (canceled)

16. (canceled)

17. A method according to claim 1, wherein the first dose of the second SARS-COV-2 vaccine is administered to the individual at least 4 months after administration of the final dose of the first SARS-COV-2 vaccine.

18. (canceled)

19. An immunogenic composition comprising mRNA encoding a single Spike (S) protein, or an immunogenic fragment or immunogenic variant thereof, derived from a first sarbecoronavirus variant, wherein the immunogenic composition is administered to an individual who has previously received one or more doses of a first SARS-COV-2 vaccine comprising or encoding an immunogen from a second sarbecoronavirus variant,
wherein the S protein is encoded as a S protein-multimerization subunit fusion, and
wherein the immunogenic composition is administered to the individual to induce an immune response against the first and second sarbecoronavirus variants and against at least a third sarbecoronavirus variant.

20. The immunogenic composition according to claim 19, wherein the S protein-multimerization subunit fusion is a protein-ferritin subunit fusion.

21. The immunogenic composition for use according to claim 19, wherein the mRNA is formulated in a lipid nanoparticle (LNP).

22. The immunogenic composition for use according to claim 19, wherein the first sarbecoronavirus variant is SARS-COV-2 Omicron.

23. (canceled)

24. (canceled)

25. (canceled)

26. The immunogenic composition according to claim 19, wherein the S protein or immunogenic fragment or immunogenic variant thereof comprises K986P and/or V987P mutations.

27. (canceled)

28. (canceled)

29. The immunogenic composition according to claim 19, wherein the first SARS-COV-2 vaccine does not comprise a nucleic acid encoding a sarbecoronavirus S protein in the form of a fusion protein that is capable of assembling to form a nanoparticle in vivo.

30. The immunogenic composition according to claim 19, wherein the third sarbecoronavirus variant is mutationally diverse from the first and/or second sarbecoronavirus variants.

31. The immunogenic composition according to claim 19, wherein the immunogenic composition is a monovalent composition.

32. The immunogenic composition according to claim 19, wherein the immunogenic composition is a bivalent composition further comprising a second mRNA encoding a single Spike (S) protein, or an immunogenic fragment or immunogenic variant thereof, wherein the S protein is encoded as a S protein-multimerization subunit fusion and is derived from a sarbecoronavirus variant that is different to said first sarbecoronavirus variant and is the same as or different to said second sarbecoronavirus variant.

33. (canceled)

34. (canceled)


SEQUENCE LISTING (18 sequences)

SEQ ID NO: 1 - RNA, 3993 nt, synthetic construct
  source 1..3993: mol_type = other RNA
atgcccctccttctccttcttcccctcctttgggctggagcgctggcctctcagtgtgtaaatctcaccacaagaacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagaggttcgacaatccagtgctgccctttaacgatggcgtgtacttcgcctccaccgagaagtctaacatcatccgcggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgggtgtattcctctgccaacaattgcacatttgagtacgtgtcccagcccttcctgatggacctggagggcaagcagggcaatttcaagaacctgcgggagttcgtgtttaagaatatcgatggctacttcaagatctactccaagcacacccccatcaacctggtgcgggacctgccacagggcttctctgccctggagccactggtggatctgcccatcggcatcaacatcacccggtttcagacactgctggccctgcacagaagctacctgacaccaggcgacagctcctctggatggaccgcaggagcagcagcctactatgtgggctatctgcagccccggaccttcctgctgaagtacaacgagaatggcaccatcacagacgccgtggattgcgccctggatcccctgtctgagacaaagtgtacactgaagagttttaccgtggagaagggcatctatcagacaagcaatttcagggtgcagcctaccgagtccatcgtgcgctttcccaatatcacaaacctgtgcccttttggcgaggtgttcaacgccaccagattcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgcagactacaattataagctgcctgacgatttcaccggctgcgtgatcgcctggaactctaacaatctggatagcaaagtgggcggcaactacaattatctgtaccggctgtttagaaagtctaatctgaagccattcgagcgggacatctccacagagatctaccaggccggctctaccccctgcaatggcgtggagggctttaactgttatttccctctgcagagctacggcttccagccaaccaacggcgtgggctatcagccctacagagtggtggtgctgtcttttgagctgctgcacgcacctgcaacagtgtgcggaccaaaaaagagcaccaatctggtgaagaacaagtgcgtgaacttcaacttcaacggcctgaccggaacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcagggcgtgaattgtaccgaggtgccagtggcaatccacgcagatcagctgacccctacatggcgggtgtactctaccggcagcaacgtgttccagacaagagccggatgcctgatcggagcagagcacgtgaacaatagctatgagtgcgacatccctatcggcgccggcatctgtgcctcctaccagacccagacaaactccccagggtctgcatcatctgtggcaagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggcagcttttgtacccagctgaatcgcgccctgacaggaatcgcagtggagcaggataagaacacacaggaggtgttcgcccaggtgaagcagatctacaagaccccacccatcaaggactttggcggcttcaatttttcccagatcctgcccgatcctagcaagccatccaagaggtcttttatcgaggacctgctgttcaacaaggtgaccctggccgatgccggcttcatcaagcagtatggcgattgcctgggcgacatcgcagcccgcgacctgatctgtgcccagaagtttaatggcctgaccgtgctgcctccactgctgacagatgagatgatcgcccagtacacatctgccctgctggcaggaaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagcagcacagcaagcgccctgggcaagctgcaggatgtggtgaatcagaacgcccaggccctgaataccctggtgaagcagctgtctagcaacttcggcgccatctcctctgtgctgaatgatatcctgagcaggctggaccctccagaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 2 - RNA, 3987 nt, synthetic construct
  source 1..3987: mol_type = other RNA
atgcccctccttctccttcttcccctcctttgggctggagcgctggcctctcagtgtgtaaatctccgtacccgaacacaactcccccccgcctataccaacagcttcacacgcggagtttactaccctgacaaggtattcaggtccagtgtactccattcaacacaggatctgttcctcccttttttcagtaatgtgacctggtttcacgctatccacgtaagcggcacgaatggcactaagaggttcgataatcctgtactgccatttaatgatggagtttacttcgcgagcatcgagaagagcaacattatccgggggtggatctttggaacaactttggacagtaaaactcaatctctccttattgttaataacgcaaccaacgtagtcatcaaggtttgcgagttccagttttgcaatgacccttttctggacgtttattatcataagaacaacaagtcatggatggaatccggcgtgtactcttccgcaaataattgtacgtttgagtatgtcagccagcctttcctcatggacctggaaggcaaacaggggaattttaaaaatctgagggagttcgtcttcaaaaatatagacgggtactttaaaatctactccaagcacacgcccatcaatttagtgagggacctgccccaaggcttttctgctcttgaaccacttgtggaccttcccatcggcatcaatatcactcgcttccagacactcttggcacttcatagatcttatctgaccccaggggacagctcctcaggttggaccgccggcgctgcagcttactacgttggatatttgcagccacgcacgtttcttctgaaatacaatgagaatggcaccattaccgacgccgtagattgcgctctcgatcctctgagcgaaaccaaatgtacattaaagagctttacggtagaaaaggggatataccaaacctctaactttcgggtccagcctaccgaatcaatcgttcggttcccaaacattaccaacttatgtccatttggagaagtgttcaatgccacgcggttcgcaagcgtttatgcctggaacagaaagcgcatctctaattgcgtggccgattactcagtcctctacaattcagctagtttcagtacatttaaatgttacggcgtgtcccccactaagttgaatgacctttgttttactaacgtctacgctgactccttcgtgatcagaggcgatgaggttcggcagatcgctcctgggcagaccggtaagatcgccgattacaactacaaactcccagacgacttcactggctgtgtgattgcctggaacagtaacaatttagactccaaagtagggggtaactacaactatcgatatagacttttccggaaaagtaacttgaaaccttttgaaagagatatatccactgaaatctatcaggccggaagtaagccctgtaatggagtcgagggcttcaattgctacttcccattacagagctatggcttccagcctacgaacggcgtcggatatcagccttaccgcgtggtggtcttgtctttcgaactgttacatgcacctgcgacagtgtgcgggcctaagaaatccaccaacctggtgaagaacaaatgcgtgaactttaatttcaacggactgactggcacaggcgttctgactgagagcaacaagaaatttctgcccttccagcaatttggccgtgacattgccgacaccacggacgctgtacgcgatccgcaaaccctggagatcctggacatcaccccatgctcttttggcggggtcagcgttattacacctgggaccaacacaagcaaccaggttgctgtcttataccagggggttaattgcaccgaagtgcctgtcgcaatccatgctgaccaacttacccccacttggcgcgtttattctaccggctccaacgtattccaaaccagggctggctgtctcatcggagcagagcatgtgaacaactcttacgaatgcgatataccaattggcgcgggaatctgtgcctcctatcagacacaaacaaacagtcgcggaagcgcgagcagtgtggcatcacagagtatcatcgcatacaccatgagccttggcgcagagaacagcgtggcctactctaataacagtattgccatacctaccaacttcaccattagcgtgacgaccgagatcctgcccgtgtctatgaccaagacatctgtggattgcacaatgtatatctgcggagacagcactgaatgctccaatctgcttttgcagtacgggtctttctgtacccagctgaatcgagcactgacaggaatcgctgtggaacaggataagaacacccaggaggtgttcgcccaggttaagcagatatacaagacccctcctataaaagacttcggtggctttaacttttctcagattctccccgacccttctaagccttctaaacgatccttcatcgaagaccttctcttcaacaaagttactttggctgatgccgggttcatcaagcagtatggtgactgtcttggcgatatcgccgccagagatttaatatgtgcccagaagttcaacggtttaacagtccttcctcctctgctgacagacgagatgattgctcagtacactagtgctctgctcgccgggactattacttcaggctggacctttggcgctggcgccgccctgcagattccctttgctatgcaaatggcataccggttcaacggtataggtgtgactcagaatgtgctgtatgaaaatcagaaactcattgcaaatcagtttaacagtgctattgggaagatacaggactccctctcttccaccgcgtccgctttaggtaagcttcaaaacgtggtaaatcaaaatgcccaggctttaaataccttggttaagcaactgagttcaaatttcggagcaatctctagcgtgcttaacgatatcctctctaggctggatccacccgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 3 - RNA, 3993 nt, synthetic construct
  source 1..3993: mol_type = other RNA
atgcccctccttctccttcttcccctcctttgggctggagcgctggcctctcagtgtgtaaatctcattacccgcacgcagctaccacctgcctatacaaactccttcacccgcggtgtctattaccccgacaaggtttttcgatcctcagttcttcattccacccaggatcttttccttccgttcttctccaacgtcacctggtttcatgctattcatgtgtcagggaccaacggaactaaaaggttcgacaatcccgtactgcctttcaacgacggggtgtatttcgcatcaacagaaaaaagcaacattatcagaggctggatttttggcactacactcgactcaaagacccaaagtttgcttattgttaataacgcaacgaatgtcgtaattaaagtctgtgaattccagttttgtaacgacccatttctggatgtatactatcataagaacaataagtcctggatggagtcagagttcagagtctactcatcagcaaacaattgcacttttgagtacgtatctcagccatttctgatggacctggaaggcaagcaagggaacttcaaaaatctccgagagtttgttttcaaaaacatagatggttacttcaagatctattctaaacacaccccgattaatcttggccgggacctgccccagggtttctctgccctcgagccccttgtagacttgcccattgggataaatattactaggtttcagacactgcttgcgctgcacaggtcatacctcactcctggggattcatcaagcggctggactgcaggtgccgctgcatactacgtgggatacctccaaccccgaacttttttgctcaagtacaatgagaatgggacgataactgatgccgtagactgcgcactcgatcctctgtctgagacgaagtgcacactgaagagtttcacggtggaaaagggtatatatcaaacatccaatttccgagtccagcccactgaaagtattgttaggttcccgaacatcacaaatctctgtccatttgacgaggtatttaacgcgacacgctttgcttccgtgtatgcgtggaatagaaagcgcattagcaactgtgtcgcggattattcagtcttgtacaactttgcgccttttttcgcatttaaatgctacggggtcagccccacaaagctgaatgatctctgcttcacaaatgtatatgcagatagctttgtcatccgaggtaacgaagtttctcagattgctcccggccaaacgggaaacatagctgactataactataagctcccagacgattttacgggttgtgtcattgcatggaactccaataaattggattcaaaggtaggtggtaattataactacaggtataggctgttcaggaagtcaaacctgaaaccgtttgaacgagatatctcaactgagatttatcaagcgggcaataaaccctgcaacggagtcgcgggtgttaattgctacttcccattgcagagttacggctttcggccgacttatggcgtcggacatcaaccttacagggtagttgttctgtctttcgaactcctgcatgctccggcaacggtgtgtggtcccaagaaaagtactaaccttgtcaaaaataagtgtgtgaacttcaattttaacggtcttacaggtactggagtattgactgagtcaaataagaagtttcttccattccaacaattcggccgcgatatagctgatacaacggatgccgtccgagaccctcagactctggagatcctggacataactccatgttcttttggaggcgttagtgtgattaccccaggcactaatacctcaaaccaagtagccgtcctgtatcaaggagtcaattgtactgaggtccctgtggccatccatgcggaccaactgacacccacatggcgcgtatatagcacaggatcaaacgtattccagactcgcgcgggttgtctgatcggggcggaatacgtaaacaatagctatgagtgtgatatacctattggagctgggatttgcgcaagctatcaaacacaaacgaaatcccacggatcggcatcatcagttgcatctcaatctattatcgcttatacaatgtctcttggcgctgagaactccgttgcttacagcaataatagcattgccataccgacgaactttactattagcgtaacgactgagatactcccggtgtctatgacaaaaactagcgttgattgtacaatgtacatttgtggggatagtacagagtgctcaaacttgcttctccagtacgggtcattctgtacccaacttaaacgcgcattgactggtattgccgtcgagcaagacaaaaacacacaagaggttttcgctcaagtaaaacaaatttacaaaacgccccctataaaatattttggcggtttcaacttttctcaaattctgcccgatccttctaagcctagtaagcgaagcttcattgaagacctgcttttcaacaaagtaaccttggctgacgcgggtttcataaaacagtatggagactgtctcggggacatagccgcaagggacttgatttgtgcccaaaaattcaacggactgacagttcttcctccacttttgactgatgagatgatagcacaatacacttctgctttgctggccggtacaataacgagtgggtggaccttcggggctggggctgccctccaaatacccttcgctatgcaaatggcatatcgatttaatggcatcggggttacacagaacgtcctgtatgaaaaccaaaagctcattgccaaccagtttaactcagcaatcggaaaaattcaggacagcctgagtagtaccgccagcgccttgggaaagttgcaggatgtcgtcaaccataacgcccaagcattgaatacgttggtcaaacagctctcctctaagttcggtgctatatcctcagtcctcaacgatatcctgagcaggctcgatccccctgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 4 - protein, 6 aa, synthetic construct
GSGGSG

SEQ ID NO: 5 - RNA, 18 nt, synthetic construct
  source 1..18: mol_type = other RNA
ggttcaggtggatcaggt

SEQ ID NO: 6 - RNA, 489 nt, synthetic construct
  source 1..489: mol_type = other RNA
gatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 7 - protein, 163 aa, synthetic construct
DIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 8 - protein, 16 aa, synthetic construct
MPLLLLLPLLWAGALA

SEQ ID NO: 9 - RNA, 4216 nt, synthetic construct
  source 1..4216: mol_type = other RNA
aggattgtgctgcatcaagcttgccgccaccatgcccctccttctccttcttcccctcctttgggctggagcgctggcctctcagtgtgtaaatctcaccacaagaacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagaggttcgacaatccagtgctgccctttaacgatggcgtgtacttcgcctccaccgagaagtctaacatcatccgcggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgggtgtattcctctgccaacaattgcacatttgagtacgtgtcccagcccttcctgatggacctggagggcaagcagggcaatttcaagaacctgcgggagttcgtgtttaagaatatcgatggctacttcaagatctactccaagcacacccccatcaacctggtgcgggacctgccacagggcttctctgccctggagccactggtggatctgcccatcggcatcaacatcacccggtttcagacactgctggccctgcacagaagctacctgacaccaggcgacagctcctctggatggaccgcaggagcagcagcctactatgtgggctatctgcagccccggaccttcctgctgaagtacaacgagaatggcaccatcacagacgccgtggattgcgccctggatcccctgtctgagacaaagtgtacactgaagagttttaccgtggagaagggcatctatcagacaagcaatttcagggtgcagcctaccgagtccatcgtgcgctttcccaatatcacaaacctgtgcccttttggcgaggtgttcaacgccaccagattcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgcagactacaattataagctgcctgacgatttcaccggctgcgtgatcgcctggaactctaacaatctggatagcaaagtgggcggcaactacaattatctgtaccggctgtttagaaagtctaatctgaagccattcgagcgggacatctccacagagatctaccaggccggctctaccccctgcaatggcgtggagggctttaactgttatttccctctgcagagctacggcttccagccaaccaacggcgtgggctatcagccctacagagtggtggtgctgtcttttgagctgctgcacgcacctgcaacagtgtgcggaccaaaaaagagcaccaatctggtgaagaacaagtgcgtgaacttcaacttcaacggcctgaccggaacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcagggcgtgaattgtaccgaggtgccagtggcaatccacgcagatcagctgacccctacatggcgggtgtactctaccggcagcaacgtgttccagacaagagccggatgcctgatcggagcagagcacgtgaacaatagctatgagtgcgacatccctatcggcgccggcatctgtgcctcctaccagacccagacaaactccccagggtctgcatcatctgtggcaagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggcagcttttgtacccagctgaatcgcgccctgacaggaatcgcagtggagcaggataagaacacacaggaggtgttcgcccaggtgaagcagatctacaagaccccacccatcaaggactttggcggcttcaatttttcccagatcctgcccgatcctagcaagccatccaagaggtcttttatcgaggacctgctgttcaacaaggtgaccctggccgatgccggcttcatcaagcagtatggcgattgcctgggcgacatcgcagcccgcgacctgatctgtgcccagaagtttaatggcctgaccgtgctgcctccactgctgacagatgagatgatcgcccagtacacatctgccctgctggcaggaaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagcagcacagcaagcgccctgggcaagctgcaggatgtggtgaatcagaacgcccaggccctgaataccctggtgaagcagctgtctagcaacttcggcgccatctcctctgtgctgaatgatatcctgagcaggctggaccctccagaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagttgatgataataggactagtggatccaactggagactgggtgaaagtgactaccagaaagtgaggaagcctaaataaacctagcgtacgtaaaaaatggaaagaacctagcgtacgaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - RNA, 4210 nt, synthetic construct
  source 1..4210: mol_type = other RNA
aggattgtgctgcatcaagcttgccgccaccatgcccctccttctccttcttcccctcctttgggctggagcgctggcctctcagtgtgtaaatctccgtacccgaacacaactcccccccgcctataccaacagcttcacacgcggagtttactaccctgacaaggtattcaggtccagtgtactccattcaacacaggatctgttcctcccttttttcagtaatgtgacctggtttcacgctatccacgtaagcggcacgaatggcactaagaggttcgataatcctgtactgccatttaatgatggagtttacttcgcgagcatcgagaaaagcaacattatccgggggtggatctttggaacaactttggacagtaaaactcaatctctccttattgttaataacgcaaccaacgtagtcatcaaggtttgcgagttccagttttgcaatgacccttttctggacgtttattatcataagaacaacaagtcatggatggaatccggcgtgtactcttccgcaaataattgtacgtttgagtatgtcagccagcctttcctcatggacctggaaggcaaacaggggaattttaaaaatctgagggagttcgtcttcaaaaatatagacgggtactttaaaatctactccaagcacacgcccatcaatttagtgagggacctgccccaaggcttttctgctcttgaaccacttgtggaccttcccatcggcatcaatatcactcgcttccagacactcttggcacttcatagatcttatctgaccccaggggacagctcctcaggttggaccgccggcgctgcagcttactacgttggatatttgcagccacgcacgtttcttctgaaatacaatgagaatggcaccattaccgacgccgtagattgcgctctcgatcctctgagcgaaaccaaatgtacattaaagagctttacggtagaaaaggggatataccaaacctctaactttcgggtccagcctaccgaatcaatcgttcggttcccaaacattaccaacttatgtccatttggagaagtgttcaatgccacgcggttcgcaagcgtttatgcctggaacagaaagcgcatctctaattgcgtggccgattactcagtcctctacaattcagctagtttcagtacatttaaatgttacggcgtgtcccccactaagttgaatgacctttgttttactaacgtctacgctgactccttcgtgatcagaggcgatgaggttcggcagatcgctcctgggcagaccggtaagatcgccgattacaactacaaactcccagacgacttcactggctgtgtgattgcctggaacagtaacaatttagactccaaagtagggggtaactacaactatcgatatagacttttccggaaaagtaacttgaaaccttttgaaagagatatatccactgaaatctatcaggccggaagtaagccctgtaatggagtcgagggcttcaattgctacttcccattacagagctatggcttccagcctacgaacggcgtcggatatcagccttaccgcgtggtggtcttgtctttcgaactgttacatgcacctgcgacagtgtgcgggcctaagaaatccaccaacctggtgaagaacaaatgcgtgaactttaatttcaacggactgactggcacaggcgttctgactgagagcaacaagaaatttctgcccttccagcaatttggccgtgacattgccgacaccacggacgctgtacgcgatccgcaaaccctggagatcctggacatcaccccatgctcttttggcggggtcagcgttattacacctgggaccaacacaagcaaccaggttgctgtcttataccagggggttaattgcaccgaagtgcctgtcgcaatccatgctgaccaacttacccccacttggcgcgtttattctaccggctccaacgtattccaaaccagggctggctgtctcatcggagcagagcatgtgaacaactcttacgaatgcgatataccaattggcgcgggaatctgtgcctcctatcagacacaaacaaacagtcgcggaagcgcgagcagtgtggcatcacagagtatcatcgcatacaccatgagccttggcgcagagaacagcgtggcctactctaataacagtattgccatacctaccaacttcaccattagcgtgacgaccgagatcctgcccgtgtctatgaccaagacatctgtggattgcacaatgtatatctgcggagacagcactgaatgctccaatctgcttttgcagtacgggtctttctgtacccagctgaatcgagcactgacaggaatcgctgtggaacaggataagaacacccaggaggtgttcgcccaggttaagcagatatacaagacccctcctataaaagacttcggtggctttaacttttctcagattctccccgacccttctaagccttctaaacgatccttcatcgaagaccttctcttcaacaaagttactttggctgatgccgggttcatcaagcagtatggtgactgtcttggcgatatcgccgccagagatttaatatgtgcccagaagttcaacggtttaacagtccttcctcctctgctgacagacgagatgattgctcagtacactagtgctctgctcgccgggactattacttcaggctggacctttggcgctggcgccgccctgcagattccctttgctatgcaaatggcataccggttcaacggtataggtgtgactcagaatgtgctgtatgaaaatcagaaactcattgcaaatcagtttaacagtgctattgggaagatacaggactccctctcttccaccgcgtccgctttaggtaagcttcaaaacgtggtaaatcaaaatgcccaggctttaaataccttggttaagcaactgagttcaaatttcggagcaatctctagcgtgcttaacgatatcctctctaggctggatccacccgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagttgatgataataggactagtggatccaactggagactgggtgaaagtgactaccagaaagtgaggaagcctaaataaacctagcgtacgtaaaaaatggaaagaacctagcgtacgaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - RNA, 4216 nt, synthetic construct
  source 1..4216: mol_type = other RNA
aggattgtgctgcatcaagcttgccgccaccatgcccctccttctccttcttcccctcctttgggctggagcgctggcctctcagtgtgtaaatctcattacccgcacgcagctaccacctgcctatacaaactccttcacccgcggtgtctattaccccgacaaggtttttcgatcctcagttcttcattccacccaggatcttttccttccgttcttctccaacgtcacctggtttcatgctattcatgtgtcagggaccaacggaactaaaaggttcgacaatcccgtactgcctttcaacgacggggtgtatttcgcatcaacagaaaaaagcaacattatcagaggctggatttttggcactacactcgactcaaagacccaaagtttgcttattgttaataacgcaacgaatgtcgtaattaaagtctgtgaattccagttttgtaacgacccatttctggatgtatactatcataagaacaataagtcctggatggagtcagagttcagagtctactcatcagcaaacaattgcacttttgagtacgtatctcagccatttctgatggacctggaaggcaagcaagggaacttcaaaaatctccgagagtttgttttcaaaaacatagatggttacttcaagatctattctaaacacaccccgattaatcttggccgggacctgccccagggtttctctgccctcgagccccttgtagacttgcccattgggataaatattactaggtttcagacactgcttgcgctgcacaggtcatacctcactcctggggattcatcaagcggctggactgcaggtgccgctgcatactacgtgggatacctccaaccccgaacttttttgctcaagtacaatgagaatgggacgataactgatgccgtagactgcgcactcgatcctctgtctgagacgaagtgcacactgaagagtttcacggtggaaaagggtatatatcaaacatccaatttccgagtccagcccactgaaagtattgttaggttcccgaacatcacaaatctctgtccatttgacgaggtatttaacgcgacacgctttgcttccgtgtatgcgtggaatagaaagcgcattagcaactgtgtcgcggattattcagtcttgtacaactttgcgccttttttcgcatttaaatgctacggggtcagccccacaaagctgaatgatctctgcttcacaaatgtatatgcagatagctttgtcatccgaggtaacgaagtttctcagattgctcccggccaaacgggaaacatagctgactataactataagctcccagacgattttacgggttgtgtcattgcatggaactccaataaattggattcaaaggtaggtggtaattataactacaggtataggctgttcaggaagtcaaacctgaaaccgtttgaacgagatatctcaactgagatttatcaagcgggcaataaaccctgcaacggagtcgcgggtgttaattgctacttcccattgcagagttacggctttcggccgacttatggcgtcggacatcaaccttacagggtagttgttctgtctttcgaactcctgcatgctccggcaacggtgtgtggtcccaagaaaagtactaaccttgtcaaaaataagtgtgtgaacttcaattttaacggtcttacaggtactggagtattgactgagtcaaataagaagtttcttccattccaacaattcggccgcgatatagctgatacaacggatgccgtccgagaccctcagactctggagatcctggacataactccatgttcttttggaggcgttagtgtgattaccccaggcactaatacctcaaaccaagtagccgtcctgtatcaaggagtcaattgtactgaggtccctgtggccatccatgcggaccaactgacacccacatggcgcgtatatagcacaggatcaaacgtattccagactcgcgcgggttgtctgatcggggcggaatacgtaaacaatagctatgagtgtgatatacctattggagctgggatttgcgcaagctatcaaacacaaacgaaatcccacggatcggcatcatcagttgcatctcaatctattatcgcttatacaatgtctcttggcgctgagaactccgttgcttacagcaataatagcattgccataccgacgaactttactattagcgtaacgactgagatactcccggtgtctatgacaaaaactagcgttgattgtacaatgtacatttgtggggatagtacagagtgctcaaacttgcttctccagtacgggtcattctgtacccaacttaaacgcgcattgactggtattgccgtcgagcaagacaaaaacacacaagaggttttcgctcaagtaaaacaaatttacaaaacgccccctataaaatattttggcggtttcaacttttctcaaattctgcccgatccttctaagcctagtaagcgaagcttcattgaagacctgcttttcaacaaagtaaccttggctgacgcgggtttcataaaacagtatggagactgtctcggggacatagccgcaagggacttgatttgtgcccaaaaattcaacggactgacagttcttcctccacttttgactgatgagatgatagcacaatacacttctgctttgctggccggtacaataacgagtgggtggaccttcggggctggggctgccctccaaatacccttcgctatgcaaatggcatatcgatttaatggcatcggggttacacagaacgtcctgtatgaaaaccaaaagctcattgccaaccagtttaactcagcaatcggaaaaattcaggacagcctgagtagtaccgccagcgccttgggaaagttgcaggatgtcgtcaaccataacgcccaagcattgaatacgttggtcaaacagctctcctctaagttcggtgctatatcctcagtcctcaacgatatcctgagcaggctcgatccccctgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagttgatgataataggactagtggatccaactggagactgggtgaaagtgactaccagaaagtgaggaagcctaaataaacctagcgtacgtaaaaaatggaaagaacctagcgtacgaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - protein, 1162 aa, synthetic construct
MPLLLLLPLLWAGALASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQSELDSIKEELDKIHKN

SEQ ID NO: 13 - protein, 1162 aa, synthetic construct
MPLLLLLPLLWAGALASQCVNLITRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQSELDSIKEELDKIHKN

SEQ ID NO: 14 - protein, 1331 aa, synthetic construct
MPLLLLLPLLWAGALASQCVNLITRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQSELDSIKEELDKIHKNGSGGSGDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 15 - RNA, 20 nt, synthetic construct
  source 1..20: mol_type = other RNA
taatacgactcactataagg

SEQ ID NO: 16 - RNA, 3981 nt, synthetic construct
  source 1..3981: mol_type = other RNA
atgcccctccttctccttcttcccctcctttgggctggagcgctggcctctcagtgtgtaaatctcattacccgcacgcagagctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagaggttcgacaatccagccctgccctttaacgatggcgtgtacttcgcctccaccgagaagtctaacatcatccgcggctggatctttggcaccacactggacagcaagacacagtccctgctgatcgtgaacaatgccaccaacgtggtcatcaaggtgtgcgagttccagttttgtaatgatccattcctggacgtgtatcagaagaacaataagtcttggatggagagcgagttccgggtgtattcctctgccaacaattgcacatttgagtacgtgtcccagcccttcctgatggacctggagggcaaggagggcaatttcaagaacctgcgggagttcgtgtttaagaatatcgatggctacttcaagatctactccaagcacacccccatcaacctggagcgggacctgccacagggcttctctgccctggagccactggtggatctgcccatcggcatcaacatcacccggtttcagacactgctggccctgcacagaagctacctgacaccagtggacagctcctctggctggaccgcaggagcagcagcctactatgtgggctatctgcagccccggaccttcctgctgaagtacaacgagaatggcaccatcacagacgccgtggattgcgccctggatcccctgtctgagacaaagtgtacactgaagagttttaccgtggagaagggcatctatcagacaagcaatttcagggtgcagcctaccgagtccatcgtgcgctttcccaatatcacaaacctgtgcccttttcacgaggtgttcaacgccaccaccttcgccagcgtgtacgcctggaataggaagcgcatctccaactgcgtggccgactattctgtgatctacaacttcgcccccttcttcgcctttaagtgctatggcgtgagccccacaaagctgaatgacctgtgctttaccaacgtgtacgccgattccttcgtgatcaggggcaacgaggtgagccagatcgcaccaggacagacaggcaacatcgcagactacaattataagctgcctgacgatttcaccggctgcgtgatcgcctggaactctaacaagctggatagcaaacccagcggcaactacaattatctgtaccggctgtttagaaagtctaagctgaagccattcgagcgggacatctccacagagatctaccaggccggcaacaagccctgcaatggcgtggccggcccaaactgttatagcccattgcagagttacggctttcggccgacttatggcgtcggacatcaaccttacagggtagttgttctgtctttcgaactcctgcatgctccggcaacggtgtgtggtcccaagaaaagtactaaccttgtcaaaaataagtgtgtgaacttcaattttaacggtcttacaggtactggagtattgactgagtcaaataagaagtttcttccattccaacaattcggccgcgatatagctgatacaacggatgccgtccgagaccctcagactctggagatcctggacataactccatgttcttttggaggcgttagtgtgattaccccaggcactaatacctcaaaccaagtagccgtcctgtatcaaggagtcaattgtactgaggtccctgtggccatccatgcggaccaactgacacccacatggcgcgtatatagcacaggatcaaacgtattccagactcgcgcgggttgtctgatcggggcggaatacgtaaacaatagctatgagtgtgatatacctattggagctgggatttgcgcaagctatcaaacacaaacgaaatcccacggatcggcatcatcagttgcatctcaatctattatcgcttatacaatgtctcttggcgctgagaactccgttgcttacagcaataatagcattgccataccgacgaactttactattagcgtaacgactgagatactcccggtgtctatgacaaaaactagcgttgattgtacaatgtacatttgtggggatagtacagagtgctcaaacttgcttctccagtacgggtcattctgtacccaacttaaacgcgcattgactggtattgccgtcgagcaagacaaaaacacacaagaggttttcgctcaagtaaaacaaatttacaaaacgccccctataaaatattttggcggtttcaacttttctcaaattctgcccgatccttctaagcctagtaagcgaagcttcattgaagacctgcttttcaacaaagtaaccttggctgacgcgggtttcataaaacagtatggagactgtctcggggacatagccgcaagggacttgatttgtgcccaaaaattcaacggactgacagttcttcctccacttttgactgatgagatgatagcacaatacacttctgctttgctggccggtacaataacgagtgggtggaccttcggggctggggctgccctccaaatacccttcgctatgcaaatggcatatcgatttaatggcatcggggttacacagaacgtcctgtatgaaaaccaaaagctcattgccaaccagtttaactcagcaatcggaaaaattcaggacagcctgagtagtaccgccagcgccttgggaaagttgcaggatgtcgtcaaccataacgcccaagcattgaatacgttggtcaaacagctctcctctaagttcggtgctatatcctcagtcctcaacgatatcctgagcaggctcgatccccctgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagt

SEQ ID NO: 17 - RNA, 4201 nt, synthetic construct
  source 1..4201: mol_type = other RNA
aggattgtgctgcatcaagcttgccgccaccatgcccctccttctccttcttcccctcctttgggctggagcgctggcctctcagtgtgtaaatctcattacccgcacgcagagctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagaggttcgacaatccagccctgccctttaacgatggcgtgtacttcgcctccaccgagaagtctaacatcatccgcggctggatctttggcaccacactggacagcaagacacagtccctgctgatcgtgaacaatgccaccaacgtggtcatcaaggtgtgcgagttccagttttgtaatgatccattcctggacgtgtatcagaagaacaataagtcttggatggagagcgagttccgggtgtattcctctgccaacaattgcacatttgagtacgtgtcccagcccttcctgatggacctggagggcaaggagggcaatttcaagaacctgcgggagttcgtgtttaagaatatcgatggctacttcaagatctactccaagcacacccccatcaacctggagcgggacctgccacagggcttctctgccctggagccactggtggatctgcccatcggcatcaacatcacccggtttcagacactgctggccctgcacagaagctacctgacaccagtggacagctcctctggctggaccgcaggagcagcagcctactatgtgggctatctgcagccccggaccttcctgctgaagtacaacgagaatggcaccatcacagacgccgtggattgcgccctggatcccctgtctgagacaaagtgtacactgaagagttttaccgtggagaagggcatctatcagacaagcaatttcagggtgcagcctaccgagtccatcgtgcgctttcccaatatcacaaacctgtgcccttttcacgaggtgttcaacgccaccaccttcgccagcgtgtacgcctggaataggaagcgcatctccaactgcgtggccgactattctgtgatctacaacttcgcccccttcttcgcctttaagtgctatggcgtgagccccacaaagctgaatgacctgtgctttaccaacgtgtacgccgattccttcgtgatcaggggcaacgaggtgagccagatcgcaccaggacagacaggcaacatcgcagactacaattataagctgcctgacgatttcaccggctgcgtgatcgcctggaactctaacaagctggatagcaaacccagcggcaactacaattatctgtaccggctgtttagaaagtctaagctgaagccattcgagcgggacatctccacagagatctaccaggccggcaacaagccctgcaatggcgtggccggcccaaactgttatagcccattgcagagttacggctttcggccgacttatggcgtcggacatcaaccttacagggtagttgttctgtctttcgaactcctgcatgctccggcaacggtgtgtggtcccaagaaaagtactaaccttgtcaaaaataagtgtgtgaacttcaattttaacggtcttacaggtactggagtattgactgagtcaaataagaagtttcttccattccaacaattcggccgcgatatagctgatacaacggatgccgtccgagaccctcagactctggagatcctggacataactccatgttcttttggaggcgttagtgtgattaccccaggcactaatacctcaaaccaagtagccgtcctgtatcaaggagtcaattgtactgaggtccctgtggccatccatgcggaccaactgacacccacatggcgcgtatatagcacaggatcaaacgtattccagactcgcgcgggttgtctgatcggggcggaatacgtaaacaatagctatgagtgtgatatacctattggagctgggatttgcgcaagctatcaaacacaaacgaaatcccacggatcggcatcatcagttgcatctcaatctattatcgcttatacaatgtctcttggcgctgagaactccgttgcttacagcaataatagcattgccataccgacgaactttactattagcgtaacgactgagatactcccggtgtctatgacaaaaactagcgttgattgtacaatgtacatttgtggggatagtacagagtgctcaaacttgcttctccagtacgggtcattctgtacccaacttaaacgcgcattgactggtattgccgtcgagcaagacaaaaacacacaagaggttttcgctcaagtaaaacaaatttacaaaacgccccctataaaatattttggcggtttcaacttttctcaaattctgcccgatccttctaagcctagtaagcgaagcttcattgaagacctgcttttcaacaaagtaaccttggctgacgcgggtttcataaaacagtatggagactgtctcggggacatagccgcaagggacttgatttgtgcccaaaaattcaacggactgacagttcttcctccacttttgactgatgagatgatagcacaatacacttctgctttgctggccggtacaataacgagtgggtggaccttcggggctggggctgccctccaaatacccttcgctatgcaaatggcatatcgatttaatggcatcggggttacacagaacgtcctgtatgaaaaccaaaagctcattgccaaccagtttaactcagcaatcggaaaaattcaggacagcctgagtagtaccgccagcgccttgggaaagttgcaggatgtcgtcaaccataacgcccaagcattgaatacgttggtcaaacagctctcctctaagttcggtgctatatcctcagtcctcaacgatatcctgagcaggctcgatccccctgaggccgaagtgcagatcgatcggctgatcactggtaggctgcagagtctgcagacatatgtcacccagcagctgatcagagcagccgagattcgagcttcagctaacttagctgccacaaagatgagtgagtgcgtgctgggccagtccaagagggtggatttctgcggaaaaggctatcatctcatgtccttcccccaaagcgcaccccacggggttgtcttcctgcacgtaacgtatgtgcccgctcaggagaagaactttactactgcccccgccatctgccacgatgggaaagctcacttcccgagagaaggagtattcgtttccaatgggacgcattggttcgtcacccagcggaacttttacgaaccacagatcattaccacggataatacatttgtgagcgggaattgcgatgtggtgattggaattgtaaataacacagtgtacgatccactgcagtcagaattagactcaattaaggaggagttggataagattcataaaaatggttcaggtggatcaggtgatatagaaaaactcctcaatgaacaagtaaataaggagatgcaaagttctaacctgtacatgagcatgtcttcttggtgttacacccatagcctcgatggagcgggattgttcctttttgaccacgctgcggaggagtatgagcatgctaaaaagctgataatatttctcaacgagaataatgttccagtgcaattgacaagtatatccgcccctgagcataagtttgaagggctcacacaaattttccaaaaggcatacgaacacgaacagcacattagcgagtctattaacaacattgttgatcatgcaatcaagtccaaagatcacgccacgtttaatttcctccagtggtatgtagctgagcaacatgaggaagaagtgttgtttaaggatattcttgataaaattgaacttattggaaatgagaaccatggcctctatcttgcggaccaatacgtcaagggaattgccaagtcccgcaagagttgatgataataggactagtggatccaactggagactgggtgaaagtgactaccagaaagtgaggaagcctaaataaacctagcgtacgtaaaaaatggaaagaacctagcgtacgaaaaaaaaaaaaaaaaaaaaaaaaaaaaaaaaaaaaaaaaaaaaaaaaaaaaaaaaaaaaaaaaaaaaaaaaaa

SEQ ID NO: 18 - protein, 1327 aa, synthetic construct
MPLLLLLPLLWAGALASQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQSELDSIKEELDKIHKNGSGGSGDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS